(19) United States
(12) Patent Application Publication
(10) Pub. No.: US 2004/0127718 A1
(43) Pub. Date: Jul. 1, 2004

(54) Substituted imidazoles

(75) Inventors: Klaus Rudolf, Warthausen (DE); Rudolf Hurnaus, Biberach (DE); Dirk Stenkamp, Biberach (DE); Stephan Mueller, Warthausen (DE); Bernd Krist, Wien (AT)

Correspondence Address: BOEHRINGER INGELHEIM CORPORATION, 900 RIDGEBURY ROAD, P. O. BOX 368, RIDGEFIELD, CT, 06877, US

(73) Assignee: Novo Nordisk A/S, Bagsvaerd (DK)

(21) Appl. No.: 10/741,880

(22) Filed: Dec. 19, 2003

Related U.S. Application Data
(63) Continuation of application No. 10/290,615, filed on Nov. 8, 2002, now abandoned.
(60) Provisional application No. 60/334,209, filed on Nov. 29, 2001.

(30) Foreign Application Priority Data
Nov. 9, 2001 (DE) 101 55 202

(51) Int. Cl. C07D 233/54

(52) U.S. Cl. 548/311.1; 548/341.1

(57) ABSTRACT

The present invention relates to novel substituted imidazoles, to the use of these compounds as medicaments, to pharmaceutical compositions comprising the compounds, and to a method of treatment employing these compounds and compositions. The present compounds show a high and selective binding affinity to the histamine H3 receptor indicating a histamine H3 receptor antagonistic or agonistic activity. As a result, the compounds are useful for the treatment of disorders related to the histamine H3 receptor. More particularly, the present compounds possess a histamine H3 receptor agonistic activity and are accordingly useful in the treatment of disorders in which a histamine H3 receptor activation is beneficial.

### RELATED APPLICATIONS

This application is a continuation of U.S. Non-Provisional application Ser. No. 10/290,615, filed Nov. 8, 2002, which claims benefit of U.S. Provisional Application Serial No. 60/334,209, filed on Nov. 29, 2001, both of which applications are herein incorporated by reference.

### FIELD OF THE INVENTION

The present invention relates to novel substituted imidazoles, to the use of these compounds as medicaments, to pharmaceutical compositions comprising the compounds, and to a method of treatment employing these compounds and compositions. The present compounds show a high and selective binding affinity to the histamine H3 receptor indicating a histamine H3 receptor antagonistic or agonistic activity. As a result, the compounds are useful for the treatment of disorders related to the histamine H3 receptor. More particularly, the present compounds possess a histamine H3 receptor agonistic activity and are accordingly useful in the treatment of disorders in which a histamine H3 receptor activation is beneficial.

### BACKGROUND OF THE INVENTION

The existence of the histamine H3 receptor has been known for several years and of current interest for the development of new medicaments (see e.g. Stark, H.; Schlicker, E.; Schunack, W., Drugs Fut. 1996, 21, 507-520; Leurs, R.; Timmerman, H.; Vollinga, R. C., Progress in Drug Research 1995, 45, 107-165). Recently, the histamine H3 receptor has been cloned, cf. Lovenberg, T. W. et al, Molecular Pharmacology, June 1999, 55, 1101-1107. The histamine H3 receptor is a presynaptic autoreceptor located in both the central and the peripheral nervous system, the skin and in organs such as the lung, the intestine, probably the spleen and the gastrointestinal tract. The histamine H3 receptor has been demonstrated to regulate the release of histamine and also of other neurotransmitters such as serotonin and acetylcholine. Accordingly, the histamine H3 receptor is an important target for new therapeutics.

H3 agonists can inhibit the release of calcitonin-gene-related peptid (CGRP) from sensory C fibres (M. Imamura, Circ. Res., 78, 1996, 863-869). Thus H3 agonists are especially useful for the treatment and prevention of diseases related to elevated CGRP levels and for the treatment and prevention of inflammatory diseases, such as ischemic arrhythmias (Silver, R. B. et al., Proc. Natl. Acad. Sci. U.S.A., 98(5), 2001, 2855-2859), myocardial ischemia and infarction (Expert Opin. Invest. Drugs (2000), 9(11), 2537-2542), migraine and asthma (Curr. Opin. Invest. Drugs (2000), 1(1), 86-89).

Furthermore H3 agonists have been reported to be of therapeutic use in dyskinesia (WO 0130346), chronic vasomotor rhinitis and as analgesics (Bulg. Chem. Commun. (2001), 33(1), 119-125/A. Rouleau, Pharmacol. Exp. Ther. (2000), 295(1), 219-225) or as gastroprotective drugs (G. Bertaccini, Dig. Dis. Sci., 40, 1995, 2052-2063).

Known H3 agonists, such as R-alpha-methylhistamine are frequently positively charged under physiological conditions (e.g. EP 0420396 A2). This property limits the use of such compounds as oral available drugs and has to be overcome for therapeutic purposes by e.g. an administration as a prodrug. The H3 agonists being subject of this patent, are void of basic aliphatic amines and of positive charges under physiological conditions and are superior compared to known H3 agonists in this respect.

Furthermore a multitude of H3 ligands, e.g. the agonist thioperamide (Br. J. Pharmacol. (1996), 118(8), 2045-2052) or the agonist histamine (Semin. Cancer Biol. (2000), 10(1), 47-53) interact with P450 isoenzymes implicating the problem of drug interactions upon therapeutic use. The H3 agonists being subject of this invention show significantly reduced interactions with P450 isoenzymes and are therefore superior compared to known H3 agonists, too.

Imidazoles similar to the compounds of the present invention have previously been prepared, and their biological properties have been investigated. Thus, WO 93/14070 and WO 96/29315 relates to monosubstituted imidazole derivatives and their use as H3 receptor antagonists.Ciproxyfan and lodoproxyfan, imidazole derivatives described in this context, have been characterized as potent histamine H3 receptor antagonists by X. Ligneau et al. (J. Pharm. And Exp. Therapeutics, 287, 1998, 658-666 and J. Pharmacol. Exp. Ther. (1994), 271(1), 452-9 respectively).

Imidazoles containing substituents bearing a sulfonamide functionality have been described in WO 97/29092, WO 99/05115, imidazoles containing substituents bearing a sulfonamide functionality or a sulfon linker have been described in WO 99/05114 and imidazoles containing substituents bearing sulfonurea linkers have been described in W099/05141. Examples amongst these compounds have been described as histamine H3 receptor ligands and more specifically as histamine H3 receptor antagonists.

Imidazoles, being linked to a piperidine ring via one of the piperidines carbon atoms, have been subject of EP 0 197 840 and of EP 0 494 010. Amongst them GT2016 has been characterized in detail as an histamine H3 receptor antagonist by C. E. Tedford et al. (J. Pharmacol. Exp. Ther. (1995), 275(2), 598-604).

Several publications disclose the preparation and use of histamine H3 agonists and antagonists. Thus, U.S. Pat. No. 4,767,778 (corresponding to EP 214 058), EP 338 939, EP 0 339 208, EP 0 387 431, EP 531 219, EP 458 661, EP 0 680 960, EP 0 717 037, WO 91/17146, WO 93/12108, WO 93/12107, WO 93/12093, WO 93/20061, U.S. Pat. No. 5,578,616 (corresponding to WO 95/14007), WO 94/17058, WO 96/38142, WO 96/38141, WO 95/11894, WO 95/14007, WO 93/20061, WO96/40126, W095/06037, WO 92/15567, WO 99/24405, WO 99/24406, WO 99/24421, WO 99/31089, WO 99/06377, U.S. Pat. Nos. 5,652,258, U.S. Pat. No. 5,837,718 and WO 94/17058 disclose imidazole derivatives having histamine H3 receptor agonistic or antagonistic activity.

Histamine receptor H3 agonists have been disclosed in EP 0 420 396, EP 0 214 058, EP 0 338 939, JP06345642 and WO 91/17146.

However, the structures of the above summarized imidazole derivatives are quite different from that of the present compounds. Thus, none of the imidazole derivatives disclosed in these publications have a second substituent at position 5 of the imidazol group such as is the case in the present compounds.

In view of the art's interest in histamine H3 receptor ligands, namely agonists and antagonists, novel compounds which interact with the histamine H3 receptor would be a highly desirable contribution to the art. The present invention provides such a contribution to the art being based on the finding that a specific class of substituted imidazole compounds displays a high and specific agonism at the histamine H3 receptor.

### DETAILED DESCRIPTION OF THE INVENTION

The present invention relates to novel, substituted imidazoles of general formula

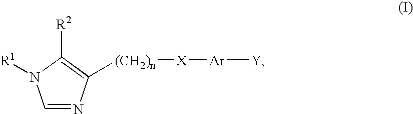

wherein

R¹ is a hydrogen atom or a functional group which can be converted into a hydrogen atom in vivo,

R² is a C₁₋₆-alkyl, C₃₋₇-cycloalkyl, aryl or aryl-C₁₋₂-alkyl group,

n is 2, 3, 4 or 5,

X is an oxygen or sulfur atom or a —CO—, —O—CH₂— or —SO—CH₂— group,

Ar is a phenylene or naphthylene group,

a 5-membered heteroarylene group linked via a carbon or nitrogen atom containing

an imino group optionally substituted by an C₁₋₄-alkyl or C₁₋₄-alkyl-carbonyl group, an oxygen or sulfur atom,

an imino group optionally substituted by an C₁₋₄-alkyl group or an oxygen or sulfur atom and additionally a nitrogen atom,

an imino group optionally substituted by an C₁₋₄-alkyl group and two nitrogen atoms or

an oxygen or sulfur atom and two nitrogen atoms,

or a 6-membered heteroarylene group containing one or two nitrogen atoms,

while the above-mentioned phenylene or 5- or 6-membered heteroarylene groups are optionally condensed via pairs of two adjacent carbon atoms with one or two saturated, unsaturated or aromatic carbocyclic or heterocyclic groups, which are optionally substituted by one or two carbonyl or C₁₋₃-alkyl groups,

and the resulting condensed bi- or tricycles may be linked to X via the carbocyclic or heterocyclic moiety, and

Y is a hydrogen, fluorine, chlorine, bromine or iodine atom, a hydroxy, cyano, C₁₋₆-alkyl, C₃₋₇-cycloalkyl, acetylene, C₁₋₄-alkyl-acetylene, C₁₋₄-alkyl-carbonyl, C₃₋₇-cycloalkyl-carbonyl, —C(═N—OH)—CH₃, phenyl, 5- or 6-membered heteroaryl, C₁₋₆-alkyloxy or phenyloxy group,

whilst the phenyl rings contained in all the above definitions may additionally be substituted by one or two fluorine, chlorine, bromine or iodine atoms, or by one or two C₁₋₆-alkyl or C ₁₋₆-alkoxy groups, while the substituents may be the same or different, and

the hydrogen atoms of alkyl groups contained in the above definitions may be partly or fully replaced by fluorine atoms,

and the diastereomers, enantiomers, mixtures and salts thereof, particularly the pharmaceutically acceptable salts thereof.

The compounds of general formula I, wherein Ar is a bond or wherein Y is one of the above-mentioned alkylsulfonyl, tosyl of silanyl groups, are usefull intermediates for the preparation of the pharmaceutically active compounds of general formula I.

The above definition of R¹ includes a functional group which can be converted into a hydrogen atom in vivo. This functional is in fact a prodrug group of the imino group. Such groups are for instance described in WO 98/46576 and by N. M. Nielsen et al. in the International Journal of Pharmaceutics 1987, 39, 75 to 85.

Examples for a group cleavable in vivo to form a imino group, i.e. a prodrug group of the imino group, are a hydroxy group, a trityl group, an acyl group like a phenylcarbonyl group optionally mono- or disubstituted by fluorine, chlorine, bromine or iodine atoms, by C₁₋₃ alkyl or C₁₋₃ alkoxy groups, while the substituents may be the same or different, a pyridinoyl group or an C₁₋₆ alkynoyl group like the formyl, acetyl, propionyl, butanoyl, pentanoyl or hexanoyl group, a 3,3,3-trichloropropionyl or allyloxycarbonyl group, a C₁₋₁₆ alkoxycarbonyl or C₁₋₁₆ alkylcarbonyloxy group wherein the hydrogen atoms may be all or partly replaced by fluorine or chlorine atoms, like the methoxycarbonyl, ethoxycarbonyl, propoxycarbonyl, isopropoxycarbonyl, butoxycarbonyl, tert.-butoxycarbonyl, pentoxycarbonyl, hexoxycarbonyl, octyloxycarbonyl, nonyloxycarbonyl, decyloxycarbonyl, undecyloxycarbonyl, dodecyloxycarbonyl, hexadecyloxycarbonyl, methylcarbonyloxy, ethylcarbonyloxy, 2,2,2-trichloroethylcarbonyloxy, propylcarbonyloxy, isopropylcarbonyloxy, butylcarbonyloxy, tert.-butylcarbonyloxy, pentylcarbonyloxy, hexylcarbonyloxy, octylcarbonyloxy, nonylcarbonyloxy, decylcarbonyloxy, undecylcarbonyloxy, dodecylcarbonyloxy or hexadecylcarbonyloxy group, a phenyl-C₁₋₆-alkoxycarbonyl group like the benzyloxycarbonyl, phenylethoxycarbonyl or phenylpropoxycarbonyl group, a 3-amino-propionyl group, in which the amino group is optionally mono- or disubstituted by C₁₋₆ alkyl or C₃₋₇ cycloalkyl groups, while the substituents may be the same or different, an C₁₋₃-alkylsulfonyl-C₂₋₄-alkoxycarbonyl, C₁₋₃-alkoxy-C₂₋₄-alkoxy-C₂₋₄-alkoxycarbonyl, Rₚ—CO—O—(R_{q}CRᵣ)—O—CO—, C₁₋₆-alkyl-CO—NH—(RₛCRₜ)—O—CO— or C₁₋₆-alkyl-O—CO—(RₛCRₜ)—(RₛCRₜ)—O—CO— group, wherein

Rₚ is a C₁₋₈ alkyl, C₅₋₇ cycloalkyl, C₁₋₈ alkyloxy, C₅₋₇ cycloalkyloxy, phenyl or phenyl-C₁₋₃-alkyl group,

R_{q} is a hydrogen atom, a C₁₋₃ alkyl, C₅₋₇ cycloalkyl or phenyl group,

Rᵣ is a hydrogen atom or a C₁₋₃ alkyl group and

Rₛ and Rₜ, which may be the same or different, are each a hydrogen atom or a C₁₋₃ alkyl group.

Alkyl and alkoxy groups mentioned in the definitions above and below include straight-chained and branched alkyl groups, such as methyl, ethyl, n-propyl, isopropyl, n-butyl, isobutyl, tert.-butyl, methoxy, ethoxy, n-propoxy, isopropoxy, n-butoxy or tert.-butoxy groups. The hydrogen atoms of alkyl or alkoxy groups are optionally partly or fully replaced by fluorine atoms, like in the trifluormethyl or trifluormethoxy group.

Cycloalkyl groups are defined as cyclic alkyl groups. Examples for cycloalkyl groups are the cyclopropyl, cyclobutyl, cyclopentanyl, cyclohexanyl and cycloheptanyl group.

An aryl group means, alone or in combination with other groups, a phenyl or naphthyl group optionally mono-, di- or trisubstituted by fluorine, chlorine, bromine or iodine atoms or C₁₋₄ alkyl or C₁₋₃ alkoxy groups, while the substituents may be the same or different.

A 5-membered heteroaryl group is a 5-membered aromatic group containing

an imino group optionally substituted by an C₁₋₄-alkyl or C₁₋₄-alkyl-carbonyl group, an oxygen or sulfur atom,

an imino group optionally substituted by an C₁₋₄-alkyl group or an oxygen or sulfur atom and additionally a nitrogen atom,

an imino group optionally substituted by an C₁₋₄-alkyl group and two nitrogen atoms or

an oxygen or sulfur atom and two nitrogen atoms.

According to the invention, a 6-membered heteroaryl group is a 6-membered aromatic group containing one or two nitrogen atoms.

A preferred embodiment according to the invention concerns compounds of general formula I, wherein

R¹ is a hydrogen atom or a trityl group,

R² is a C₁₋₄-alkyl, C₃₋₅-cycloalkyl or aryl group,

n is 2, 3 or 4,

X is an oxygen or sulfur atom or a —O—CH₂— or —SO—CH₂— group,

Ar is a 1,2-phenylene, 1,3-phenylene, 1,4-phenylene, 2,5-naphthylene or 2,6-naphthylene group,

a 5-membered heteroarylene group linked via a carbon or nitrogen atom and a carbon atom containing

an imino group optionally substituted by an C₁₋₄-alkyl or C₁₋₄-alkyl-carbonyl group, an oxygen or sulfur atom,

an imino group optionally substituted by an C₁₋₄-alkyl group or an oxygen or sulfur atom and additionally a nitrogen atom,

an imino group optionally substituted by an C₁₋₄-alkyl group and two nitrogen atoms or

an oxygen or sulfur atom and two nitrogen atoms,

or a 6-membered heteroarylene group containing one or two nitrogen atoms,

while two adjacent carbon atoms of the above-mentioned phenylene or 5- or 6-membered heteroarylene groups are optionally bridged by a —CH₂—CH₂—CH₂—CH₂—, —(C═O)—CH₂—CH₂—CH₂—, —C(CH₃)₂—CH₂—CH₂—C(CH₃)₂—, —CH═CH—CH═N—, —O—CH₂—O— or —N═CH—S— group

and the resulting bicycles are linked to X via the carbocyclic moiety, and

Y is a hydrogen, fluorine, chlorine, bromine or iodine atom, a hydroxy, cyano, C₁₋₄-alkyl, C₃₋₅-cycloalkyl, acetylene, C₁₋₄-alkyl-acetylene, C₁₋₄-alkyl-carbonyl, C₃₋₅-cycloalkyl-carbonyl, phenyl, —C(═N—OH)—CH₃, C₁₋₆-alkyloxy, phenyloxy group or a 5- or 6-membered heteroaryl group as defined above,

whilst the phenyl rings contained in all the above definitions may additionally be substituted by one or two halogen atoms, C₁₋₆-alkyl or C₁₋₆-alkoxy groups while the substituents may be the same or different and

the hydrogen atoms of alkyl groups contained in the above definitions may be partly or fully replaced by fluorine atoms,

and the diastereomers, enantiomers, mixtures and salts thereof, particularly the pharmaceutically acceptable salts thereof,

particularly those compounds, wherein X is an oxygen or sulfur atom or a —O—CH₂— group.

Another preferred embodiment of the invention concerns compounds of general formula I, wherein

R¹ is a hydrogen atom,

R² is a C₁₋₄-alkyl, C₃₋₅-cycloalkyl or phenyl group,

n is 2, 3 or 4,

X is an oxygen atom or a —O—CH₂— group,

Ar is a group selected from the formulae

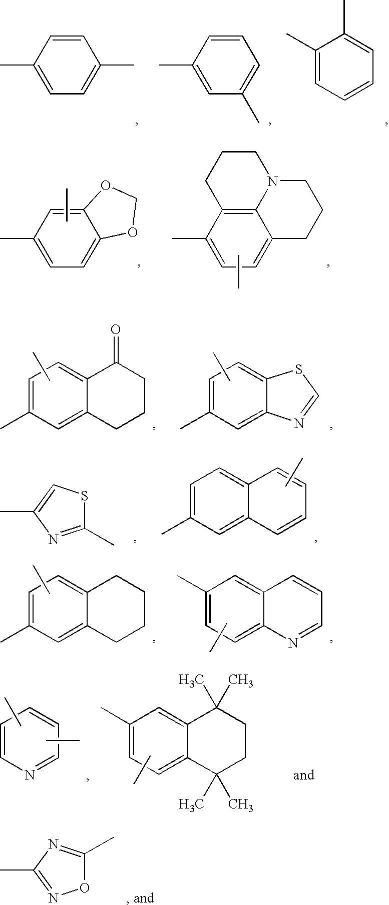

Y is a hydrogen, fluorine, chlorine, bromine or iodine atom, a hydroxy, cyano, C₁₋₄-alkyl, C₃₋₅-cycloalkyl, acetylene, C₁₋₄-alkyl-carbonyl, C₃₋₅-cycloalkyl-carbonyl, phenyl, —C(═N—OH)—CH₃, C₁₋₃-alkoxy, phenyloxy or imidazolyl group,

whilst the phenyl rings contained in all the above definitions may additionally be substituted by a halogen atom, a C₁₋₃-alkyl or C₁₋₃-alkoxy group and

the hydrogen atoms of alkyl groups contained in the above definitions may be partly or fully replaced by fluorine atoms,

and the diastereomers, enantiomers, mixtures, prodrugs and salts thereof, particularly the pharmaceutically acceptable salts thereof.

Still another preferred embodiment of the invention concerns compounds of general formula I, wherein

R¹ is a hydrogen atom,

R² is a methyl, ethyl or isopropyl group,

n is 2, 3 or 4,

X is an oxygen atom or a —O—CH₂— group,

Ar is a 1,3- or 1,4-phenylene or 2,5-napthylene group and

Y is a hydrogen, fluorine, chlorine, bromine or iodine atom, a hydroxy, cyano, C₁₋₄-alkyl, acetylene, C₁₋₄-alkyl-carbonyl, C₃₋₅-cycloalkyl-carbonyl, phenyl, C₁₋₃-alkoxy, phenoxy or imidazolyl group,

whilst the phenyl rings contained in all the above definitions may additionally be substituted by a fluorine, chlorine, bromine or iodine atom atom, a C₁₋₃-alkyl or C₁₋₃-alkoxy group and

the hydrogen atoms of alkyl groups contained in the above definitions may be partly or fully replaced by fluorine atoms,

and the diastereomers, enantiomers, mixtures, prodrugs and salts thereof, particularly the pharmaceutically acceptable salts thereof,

most particularly those compounds, wherein

R² is a methyl group

and the diastereomers, enantiomers, mixtures, prodrugs and salts thereof, particularly the pharmaceutically acceptable salts thereof.

Preferred compounds of general formula I are selected from the group consisting of

(a) 5-Methyl-4-[4-(naphthalen-2-yloxy)-butyl]-1H-imidazole,

(b) 4-[3-(4-Iodo-benzyloxy)-propyl]-5-methyl-1H-imidazole,

(c) 5-Methyl-4-[3-(4-trifluoromethoxy-benzyloxy)-propyl]-1H-imidazole,

(d) 5-Methyl-4-[3-(naphthalen-2-ylmethoxy)-propyl]-1H-imidazole,

(e) 5-Methyl-4-[3-(4-trifluoromethyl-benzyloxy)-propyl]-1H-imidazole,

(f) 4-[3-(3,5-Dichloro-benzyloxy)-propyl]-5-methyl-1H-imidazole,

(g) 4-[3-(3,5-Bis-trifluoromethyl-benzyloxy)-propyl]-5-methyl-1H-imidazole,

(h) 4-[3-(3-Iodo-benzyloxy)-propyl]-5-methyl-1H-imidazole,

(i) 5-Methyl-4-[3-(3-trifluoromethyl-benzyloxy)-propyl]-1H-imidazole,

(j) 4-[2-(4-Iodo-benzyloxy)-ethyl]-5-methyl-1H-imidazole,

(k) 5-Methyl-4-[4-(4-trifluoromethoxy-benzyloxy)-butyl]-1H-imidazole and

(i) 4-[3-(3,5-Dimethyl-benzyloxy)-propyl]-5-methyl-1H-imidazole,

and the diastereomers, enantiomers, mixtures and salts thereof, particularly the pharmaceutically acceptable salts thereof.

A process for preparing the compounds according to the invention is characterised in that

a) in order to prepare a compound of general formula I wherein X denotes an oxygen atom:

a compound of general formula

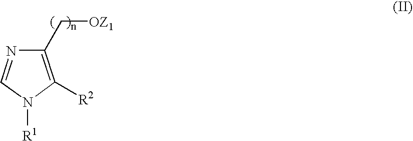

optionally formed in the reaction mixture,

wherein

R¹ and R² are as defined above and

Z₁ denotes a leaving group such as tosylate, an C₁₋₄-alkyl sulfonate or the like,

is etherified with an alcohol of general formula

HO—Ar—Y (III),

wherein Ar and Y are as defined above,

under basic conditions or

b) in order to prepare a compound of general formula I wherein X denotes an oxygen atom:

a compound of general formula

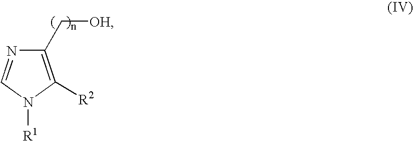

optionally formed in the reaction mixture,

wherein

R¹ and R² are as defined above,

is etherified with an alcohol of general formula

HO—Ar—Y (III),

wherein Ar and Y are as defined above,

in the presence of triphenylphosphine and diethyl azodicarboxylate or

c) in order to prepare a compound of general formula I wherein X denotes an —O—CH₂— group:

a compound of general formula IV optionally formed in the reaction mixture wherein R¹ and R² are as defined above

is reacted with a compound of general formula

Z₂—CH₂—Ar—Y (V),

wherein Ar and Y are as defined above

and Z₂ denotes a leaving group such as a chlorine or bromine atom or a mesylate or tosylate group, and

any protecting group used during the reactions to protect reactive groups is cleaved and/or

the group Y, if desired, may be subsequently transformed into the desired group and/or

a compound of general formula I thus obtained is resolved into its stereoisomers and/or

a compound of general formula I thus obtained is converted into its salts, particularly, for pharmaceutical use, into the physiologically acceptable salts thereof with an inorganic or organic acid or base.

Synthetic routes towards imidazoles containing substituents at position 4 and 5 have been described starting from p-toluenesulfonylacetonitrile which can be alkylated at the methylene bridge (Convery, M. A.; Davis, A. P.; Dunne, C. J.; MacKinnon, J. W.; Tetrahedron Lett 1995, 36 (24), 4279-4282; Tsuoda, T.; Nagaku, M.; Nagino, C.; Kawamura, Y.; Ozaki, F.; Hioki, H.; Ito, S.; Tetrahedron Lett 1995, 36 (14), 2531-2534). The resulting intermediate undergoes ring closure with aldehydes under basic conditions resulting in dihydro-oxazoles (Possel, O.; Van Leusen, A. M.; Heterocycles [HTCYAM] 1977, 7, 77). These dihydro-oxazol derivatives can be transformed in a final step by treatment with ammonia to the corresponding imidazole (Home, D. A.; Yakushijin, K.; Buechi, G.; Heterocycles [HTCYAM] 1994, 39 (1), 139-153). The residue in position 4 derives in this synthetic sequence from the used alkylbromide, the residue in position 5 of this ring system derives from the used aldehyde.

Another route to imidazoles, characterized by a methyl group in position 5 and an n-alkyl group of at least two carbon atoms functionalized with a terminal hydroxy group, starts with 5-methylimidazole4-carboxyaldehyde.

Synthesis of 3-(5-methyl-1H-imidazol-4-yl)-propionic acid ester has been outlined in Bioorg. Med. Chem. Lett. (1992, 2[12], 1509-1512). This compound can be N-tritylated with tritylchloride following standard procedures. Subsequent treatment with lithium aluminium hydride yields N-trityl-3-(5-methyl-1H-imidazol-4-yl)-propan-1-ol. Substituting (carboxymethyl)-triphenylphosphonium bromide with other phosphonium compounds gives access to a variety of other chain lengths. Thus, phosphonium compounds containing protected alcoholes as well as phosphonium compounds functionalized with esters via linkers like alkyl groups or alkyene groups can be used in this synthesis.

General Procedure According to Process a)

An aromatic compound containing a phenolic hydroxy group dissolved in a suitable solvent, for instance DMSO or DMF, is converted into the corresponding phenolate by addition of a base, for instance a NaH- or KH-dispersion, at about 20° C. Subsequently an N-protected imidazole derivative functionalized with an alcohol whose hydroxy group has been transformed into a suitable leaving group, for instance into a C₁₋₄-alkyl sulfonate or tosylate group, is added in a molar ratio from 0.7 to 1.3, preferentially in substantially stoichiometric amounts, at about 20° C. and the mixture is then heated to 50 to 90° C., preferentially to 70° C., for several hours. The reaction mixture is then worked up in a suitable manner and the crude product can be purified using known methods.

General Procedure According to Process b)

A substantially stoichiometric mixture of a N-protected imidazole derivative functionalized with an aliphatic alcohol, triphenylphosphine and an aromatic compound containing a phenolic hydroxy group in a suitable solvent, for instance THF or diethylether, is treated with diethyl azodicarboxylate at about 20° C. until the reation is completed (about 10 hours to 3 days). The product is isolated and purified using well-known methods.

General Procedure According to Process c)

To a stirred solution of a N-protected imidazole derivative fuctionalized with an aliphatic alcohol in a suitable solvent, for instance THF or diethylether, a base, for example a NaH- or KH-dispersion, and an auxiliary enhancing the solubility of the base, for instance 15-crown-5, are added at about 20° C. in order to obtain the corresponding alcoholate. The reaction mixture is cooled to about 4° C. followed by addition of an benzylic electrophil, for example a benzylhalide or benzylmesylate, in a molar ratio of 0.7 to 1.3, preferentially in a substantially stoichiometric amount, and stirred at about 20° C. for another 1-24 hours. The reaction mixture is then worked up in a suitable manner and the crude product can be purified using known methods.

General Procedure for the Clevage of a Trityl Group from an Imidazole Residue:

The N-tritylated imidazole is dissolved in a suitable organic solvent, for instance THF or dioxan, and 2 N HCl (1:3) before heating to about 70° C. for 2 h. The reaction mixture is then worked up in a suitable manner, for example by removing the solvent under reduced pressure, extracting triphenylmethanol with Et₂O, neutralizing the aqueous layer with K₂CO₃ and extracting the product with Et₂O and CHCl₃. After evaporation of the solvent the combined organic extracts then give the detritylated product.

In the reactions described hereinbefore, any reactive groups present such as carboxy, hydroxy, amino, alkylamino or imino groups may be protected during the reaction by conventional protecting groups which are cleaved again after the reaction.

For example, a protecting group for a carboxyl group may be a trimethylsilyl, methyl, ethyl, tert.butyl, benzyl or tetrahydropyranyl group and

protecting groups for a hydroxy, amino, alkylamino or imino group may be an acetyl, trifluoroacetyl, benzoyl, ethoxycarbonyl, tert.butoxycarbonyl, benzyloxycarbonyl, benzyl, methoxybenzyl or 2,4-dimethoxybenzyl group and additionally, for the amino group, a phthalyl group.

Any protecting group used is optionally subsequently cleaved for example by hydrolysis in an aqueous solvent, e.g. in water, isopropanol/water, tetrahydrofuran/water or dioxan/water, in the presence of a acid such as trifluoroacetic acid, hydrochloric acid or sulphuric acid or in the presence of an alkali metal base such as lithium hydroxide, sodium hydroxide or potassium hydroxide, at temperatures between 0 and 100° C., preferably at temperatures between 10 and 50° C.

However, a benzyl, methoxybenzyl or benzyloxycarbonyl group is cleaved, for example, hydrogenolytically, e.g. with hydrogen in the presence of a catalyst such as palladium/charcoal in a solvent such as methanol, ethanol, ethyl acetate, dimethylformamide, dimethylformamide/acetone or glacial acetic acid, optionally with the addition of an acid such as hydrochloric acid or glacial acetic acid at temperatures between 0 and 50° C., but preferably at ambient temperature, and at a hydrogen pressure of 1 to 7 bar, but preferably 3 to 5 bar.

A methoxybenzyl group may also be cleaved in the presence of an oxidising agent such as cerium(IV)ammonium nitrate in a solvent such as methylene chloride, acetonitrile or acetonitrile/water at temperatures of between 0 and 50° C., but preferably at ambient temperature.

A 2,4-dimethoxybenzyl group, however, is preferably cleaved in trifluoroacetic acid in the presence of anisole.

A tert.butyl or tert.butyloxycarbonyl group is preferably cleaved by treating with an acid such as trifluoroacetic acid or hydrochloric acid, optionally using a solvent such as methylene chloride, dioxan, ethyl acetate or ether.

A phthalyl group is preferably cleaved in the presence of hydrazine or a primary amine such as methylamine, ethylamine or n-butylamine in a solvent such as methanol, ethanol, isopropanol, toluene/water or dioxan at temperatures between 20 and 50° C.

Moreover, chiral compounds of general formula I obtained may be resolved into their enantiomers and/or diastereomers.

Thus, for example, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical antipodes and compounds of general formula I with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the mixture of diastereomeric salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, N-acetylglutamic acid, aspartic acid, N-acetylaspartic acid or quinic acid. An optically active alcohol may be for example (+)- or (−)-menthol and an optically active acyl group in amides, for example, may be a (+)- or (−)-menthyloxycarbonyl group.

Furthermore, the compounds of formula I may be converted into the salts thereof, particularly for pharmaceutical use into the physiologically acceptable salts with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

Pharmacological Methods

The ability of the compounds to interact with the histamine H3 receptor was determined by the following in vitro binding assays:

Binding Assay I

Rat cerebral cortex was homogenized in ice cold K-Hepes, 5 mM MgCl₂ pH 7.1 buffer. After two differential centrifugations the last pellet was resuspended in fresh Hepes buffer containing 1 mg/mL bacitracin. Aliquots of the membrane suspension (400 mg/mL) were incubated for 60 min at 25° C. with 30 pM [¹²⁵I]-iodoproxifan, a known histamine H3 receptor antagonist, and the test compound at various concentrations. The incubation was stopped by dilution with ice-cold medium, followed by rapid filtration through Whatman GF/B filters pretreated for 1 h with 0.5% polyethyleneimine. The radioactivity retained on the filters was counted using a Cobra II auto gamma counter. The radioactivity of the filters was indirectly proportional to the binding affinity of the tested compound. The results were analyzed by nonlinear regression analysis.

Binding Assay II

The H3-receptor agonist ligand R-α-methyl[³H]histamine was incubated with isolated rat cortex cell-membranes at 25° C. for 1 h, followed by a filtration of the incubate through Whatman GF/B filters. Radioactivity retained on the filters was measured using a beta counter.

Male Wistar rats (150-200 g) were decapitated and cerebral cortex was quickly dissected out and frozen immediately on dry ice. Tissue was kept at −80° C. until membrane preparation. During the membrane preparation the tissue was kept on ice all the time. Rat cerebral cortex was homogenized in 10 volumes (w/w) ice-cold Hepes buffer (20 mM Hepes, 5 mM MgCl₂ pH 7.1 (KOH)+1 mg/ml bacitracin) using a Ultra-Turrax homogenizer for 30 seconds. The homogenate was centrifuged at 140 g in 10 min. The supernatant was transferred to a new test tube and centrifuged for 30 min at 23 000 g. Pellet was resuspended in 5-10 ml Hepes buffer, homogenized and centrifuged for 10 min at 23 000 g. This short centrifugation step is repeated twice. After the last centrifugation the pellet was resuspended in 2-4 ml Hepes buffer and the protein concentration was determined. The membranes were diluted to a protein concentration of 5 mg/ml using Hepes buffer, aliquoted and stored at −80° C. until use.

50 μL test-compound, 100 μl membrane (200 mg/ml), 300 μl Hepes buffer and 50 μl R-α-methyl[³H]histamine (1 nM) were mixed in a test tube. The compounds to be tested were dissolved in DMSO and further diluted in H₂O to the desired concentrations. Radioligand and membranes were diluted in Hepes buffer+1 mg/ml bacitracin. The mixture was incubated for 60 min at 25° C. Incubation was terminated by adding 5 ml ice-cold 0.9% NaCl, followed by rapid filtration through Whatman GF/B filters pre-treated for 1 h with 0.5% polyethyleneimine. The filters were washed with 2×5 ml ice-cold NaCl. To each filter a 3 ml scintillation cocktail was added and the radioactivity retained was measured with a Packard Tri-Carb beta counter.

IC₅₀ values were calculated by non-linear regression analysis of binding curves (6 points minimum) using the windows program GraphPad Prism, GraphPad software, USA.

When tested, the present compounds of the formula (I) generally showed a high binding affinity to the histamine H3 receptor.

Preferably, the compounds according to the invention have an IC₅₀ value as determined by one or both of the assays of less than 1 μM, more preferred of less than 500 nM and even more preferred of less than 100 nM.

Binding Assay III

The H3 receptor was cloned by PCR and subcloned into the pcDNA3 expression vector. Cells stably expressing the H3 receptor were generated by transfecting the H3-expression vectors into HEK 293 cells and using G418 to select for H3 clones. The h-H3-HEK 293 clones were cultured in DMEM with glutamax, 10% FCS, 1% Pen/Strep and 1 mg/ml G 418 at 37° C. and 5% CO₂. Before harvesting, the confluent cells were rinsed with PBS and incubated with Versene for approximately 5 minutes. The cells were flushed with PBS and DMEM and the cellsuspension collected in a tube and centrifuged for 5-10 min at 1500 rpm in a Heraeus Sepatech Megafuge 1.0. The pellet was resuspended in 10-20 vol. Hepes buffer (20 mM Hepes, 5 mM MgCl₂, pH 7.1 (KOH)) and homogenized for 10-20 seconds using a Ultra-Turrax homogenizer. The homogenate was centrifuged for 30 min at 23 000 g. The pellet was resuspended in 5-10 ml Hepes buffer, homogenized 5-10 seconds with the Ultra-Turrax and centrifuged for 10 min at 23 000 g. Following this centrifugation step, the membrane pellet was resuspended in 2-4 ml Hepes buffer, homogenized with a syringe or teflonhomogenizer, and the protein concentration determined. The membranes were diluted to a protein concentration of 1-5 mg/ml in Hepes buffer, aliquoted and kept at −80° C. until use.

Aliquots of the membrane suspension were incubated for 60 min at 25° C. with 30 pM [¹²⁵I]-iodoproxifan, a known compound with high affinity for the H3 receptor, and the test compound at various concentrations. The incubation was stopped by dilution with ice-cold medium, followed by rapid filtration through Whatman GF/B filters pretreated for 1 h with 0.5% polyethyleneimine. The radioactivity retained on the filters was counted using a Cobra II auto gamma counter. The radioactivity of the filters was indirectly proportional to the binding affinity of the tested compound. The results were analyzed by nonlinear regression analysis.

Preferably, the compounds according to the invention have an IC₅₀ value as determined by the assay of less than 1 μM, more preferred of less than 500 nM and even more preferred of less than 100 nM.

IC₅₀ of Example 15/5 (see below): <100 nM

IC₅₀ of Example 16/14 (see below): <50 nM

Furthermore, in a similar way binding assays were carried out in order to determine the ability of the present compounds to interact with the histamine HI receptor (reference compound [¹²⁵I]-pyrilamine) and the histamine H2 receptor (reference compound [¹²⁵I]-aminopotentidine), respectively. These assays showed that the present compounds do not show a high affinity for these receptors and hence are very specific to the histamine H3 receptor.

Functional Assay

The ability of the compounds to interact with the histamine H3 receptor as agonists, inverse agonists and/or competitive antagonists, was determined by an in vitro functional assay utilizing membranes from HEK293 cell expressing the human H3 receptors.

The H3 receptor was cloned by PCR and subcloned into the pcDNA3 expression vector. Cells stably expressing the H3 receptor were generated by transfecting the H3-expression vectors into HEK 293 cells and using G418 to select for H3 clones. The h-H3-HEK 293 clones were cultured in DMEM with glutamax, 10% FCS, 1% Pen/Strep and 1 mg/ml G 418 at 37° C. and 5% CO₂.

The H3 receptor expressing cells were washed once with phosphate buffered saline (PBS) and harvested using versene (GIBCO-BRL). PBS was added and the cells were centrifuged for 5 min at 188 g. The cell pellet was resuspended in stimulation buffer to a concentration of 1×10⁶ cells/ml. cAMP accumulation was measured using the Flash Plate® cAMP assay (NEN™ Life Science Products). The assay was generally performed as described by the manufacturer. Briefly, 50 μl cell suspension was added to each well of the Flashplate which also contained 25 μl 40 μM isoprenaline, to stimulate cAMP generation, and 25 μl of test compound (either agonists or inverse agonists alone, or agonist and competitive antagonist in combination). The final volume in each well was 100 μl. Test compounds were dissolved in DMSO and diluted in H₂O. The mixture was shaken for 5 minutes, and allowed to stand for 25 minutes at room temperature. The reaction was stopped with 100 μl “Detection Mix” per well. The plates were then sealed with plastic, shaken for 30 minutes, allowed to stand overnight, and finally the radioactivity was counted in the Cobra II auto gamma topcounter. EC₅₀ values were calculated by non-linear regression analysis of dose response curves (6 points minimum) using GraphPad Prism. Kb values were calculated by Schild plot analysis.

EC₅₀ of Example 15/5 (see below): <100 nM

EC₅₀ of Example 16/14 (see below): <50 nM

Pharmaceutical Compositions

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well-known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain of from 0.05 to about 1000 mg, preferably from about 0.1 to about 500 mg, and more preferred from about 0.5 mg to about 200 mg.

For parenteral routes, such as intravenous, intrathecal, intramuscular and similar administration, typically doses are in the order of about half the dose employed for oral administration.

The compounds of this invention are generally utilized as the free substance or as a pharmaceutically acceptable salt thereof. One example is an acid addition salt of a compound having the utility of a free base. When a compound according to the invention contains a free base such salts are prepared in a conventional manner by treating a solution or suspension of a free base of the compound according to the invention with a chemical equivalent of a pharmaceutically acceptable acid, for example, inorganic and organic acids. Representative examples are mentioned above. Physiologically acceptable salts of a compound with a hydroxy group include the anion of said compound in combination with a suitable cation such as sodium or ammonium ion.

For parenteral administration, solutions of the present compounds in sterile aqueous solution, aqueous propylene glycol or sesame or peanut oil may be employed. Such aqueous solutions should be suitable buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water. Similarly, the carrier or diluent may include any sustained release material known in the art, such as glyceryl monostearate or glyceryl distearate, alone or mixed with a wax. The pharmaceutical compositions formed by combining the compounds according to the invention and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. These formulations may be in the form of powder or granules, as a solution or suspension in an aqueous or non-aqueous liquid, or as an oil-in-water or water-in-oil liquid emulsion.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

If desired, the pharmaceutical composition of the invention may comprise the compound of the formula I in combination with further pharmacologically active substances.

The following examples shall illustrate the invention without limiting the scope of the invention. Examples 1, 2 and 4 to 14 describe the preparation of starting materials or intermediates. The compounds of Examples 3, 15, 16 and 17 including the compounds prepared analogously are pharamceutically active compounds according to the invention.

Abbreviations:

Ac: acetyl residue

DCM: dichloromethane, methylenechloride

DMF: N,N-dimethyl formamide

DMSO: dimethyl sulfoxide

EDC: N-ethyl-N′-(3-dimethylaminopropyl)carbodiimide hydrochloride

HOBt: N-hydroxybenzotriazole, 1-hydroxybenzotriazole

MP: melting point

NMP: N-methylpyrrolidone

SiO₂: silica

TBAF: tetrabutylammoniumfluoride

TBDMS: tert-Butyldimethylsilyl residue

THF: tetrahydrofuran

TLC: thin layer chromatography

Tosmic: p-toluenesufonylmethyl isocyanide

Tr: Trityl residue

### EXAMPLES

### Example 1

### N-Trityl-3-(5-methyl-1H-imidazol-4-yl)-propan-1-ol

Trityl-3-(5-methyl-1H-imidazol-4-yl)-propan-1-ol was synthesized according to Scheme 1 following the following procedure:

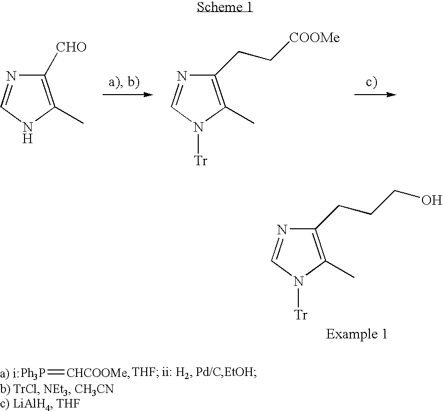

a) Preparation of 3-(3H-imidazol-4-yl)-propionic acid ester has been carried out following the synthetic procedure described in Bioorg. Med. Chem. Lett. 1992, 2(12), 1509-1512.

b) To a solution of 3-(3H-imidazol-4-yl)-propionic acid ester (97 mmol) in acetonitrile (250 ml) were added triethylamine (27 ml, 194 mmol) and a solution of tritylchloride (40.6 g, 146 mmol) in acetonitrile (500 ml). The resulting mixture was stirred at 20° C. overnight. The mixture was concentrated under reduced pressure and the residue redissolved in ethylacetate, washed with water (2×) and brine (1×) and concentrated under reduced pressure.

c) To a solution of N-trityl-3-(3H-imidazol-4-yl)-propionic acid ester (32 mmol) in THF (100 ml) was added lithium aluminium hydride (24 ml, 1 mol/l in THF, 24 mmol). The mixture was stirred at 20° C. for 45 min, and water (3.5 ml) and NaOH (35 ml, 4 mol/l in water) were carefully added. Water (14 ml) was added before neutralization of the solution with aqueous HCl, filtration and concentration under reduced pressure.

### Example 2

### 3-(5-Methyl-1H-imidazol-4-yl)-propan-1-ol hydrochloride

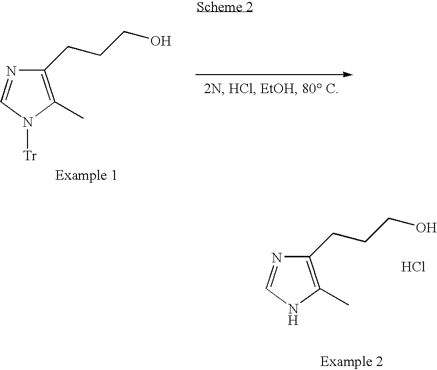

A solution of 29 mmol of example 1 in 40 ml ethanol and 130 ml 2M HCl was refluxed for 2 h and completion of the reaction was monitored by TLC. After removal of the solvent under reduced pressure, the crude product was extracted twice with ethyl acetate in order to remove tritanol and the residue dried in vacuo.

### Example 3/1

### 5-Methyl-4-(3-phenoxy-propyl)-1H-imidazole

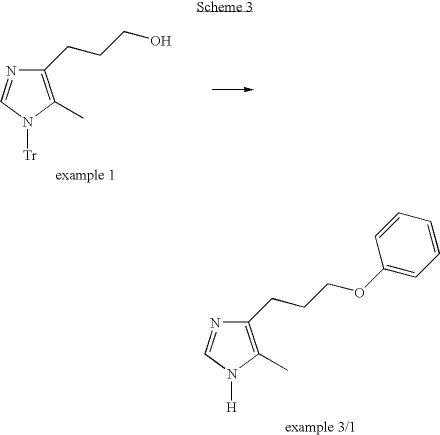

A mixture of example 1 (1.9 g, 5 mmol), triphenylphosphine (1.6 g, 6 mmol), and phenol (0.47 g, 5 mmol) was dissolved in 20 mL of freshly distilled dry THF under nitrogen and cooling. A solution of diethyl azodicarboxylate (1.1 g, 6 mmol) in 4 mL of THF was added to this mixture and the reaction mixture stirred at ambient temperature for 48 h. After removal of the solvent under reduced pressure and silica gel column chromatography using ethyl acetate, the residue was dissolved in 10 ml of THF and 30 mL of 2 N HCl. The reaction mixture was heated at 70° C. for 2 h. The solvent was evaporated under reduced pressure, and triphenylmethanol was extracted with Et₂O. The aqueous layer was neutralized with K₂CO₃ and the product extracted with Et₂O and CHCl₃. The combined organic extracts were dried and evaporated to give an oil.

| example | structure | Name |
|---|---|---|
| 3/1 | 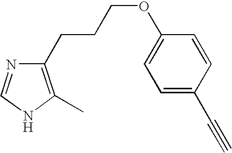 | 5-Methyl-4-(3-phenoxy- propyl)-1H-imidazole |

Mass Spectrometry: [M + H]⁺ 217
¹H-NMR (300 MHz, CDCl₃): 2.0-2.2 (m, 5H); 2.74 (t, 2H); 3.93 (t, 2H); 6.8-6.97 (m, 3H); 7.28-7.30 (m, 2H); 7.52 (s, 1H).
TLC (solvent: DCM/MeOH = 95:5, Polygram ALOX) R_{f}: 0,50
MP: 109° C.

### Examples 3/2-3/21

Examples 3/2 to 3/22 were prepared according to the procedure described for example 3/1.

| example | structure | Name |
|---|---|---|
| 3/2 | 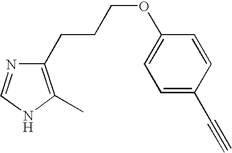 | 4-[3-(4-Ethynyl- phenoxy)-propyl]-5- methyl-1H-imidazole |

Example 3/2 has been prepared starting from example 1 and 4-ethynyl-phenol.
(4-Ethynyl-phenol has been prepared according to Cevasco, Giorgio; Pardini, Roberto; Thea, Sergio; Eur. J. Org. Chem.; 4; 1998; 665-670.)
Mass Spectrometry: [M + H ⁺ 242
Mass Spectrometry: [M − H ⁻ 239
¹H-NMR (300 MHz, CD₃OD): 2.0-2.1 (m, 5H); 2.72 (t, 2H); 3.93 (t, 2H); 6.83-6.86 (d, 2H); 7.34-7.37 (d, 2H); 7.57 (s, 1H).
TLC (solvent: DCM/MeOH = 95:5, Polygram ALOX) R_{f}: 0,47
MP: 85° C.

| example | structure | Name |
|---|---|---|
| 3/3 | 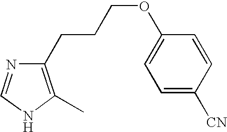 | 1-{4-[3-(5-Methyl- 1H-imidazol-4-yl)- propoxy]-phenyl}- ethanone |

Example 3/3 has been prepared starting from example 1 and 4-hydroxyacetophenone.
Mass Spectrometry: [M + H]⁺ 259
Mass Spectrometry: [M − H]⁻ 257
¹H-NMR (300 MHz, CDCl₃): 2.08-2.2 (m, 5H); 2.53 (s, 3H); 2.76 (t, 2H); 3.98 (t, 2H); 6.85-89 (d, 2H); 7.86-7.9 (d, 2H); 7.63 (s, 1H).
TLC (solvent: DCM/MeOH = 95:5, Polygram ALOX) R_{f}: 0,46
MP: 96° C.

| example | structure | Name |
|---|---|---|
| 3/4 | 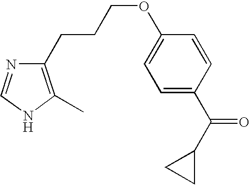 | Cyclopropyl-{4-[3- (5-methyl-1H- imidazol-4-yl)- propoxy]-phenyl}- methanone |

Example 3/4 has been prepared starting from example 1 and cyclopropyl-(4-hydroxy-phenyl)-methanone. (Cyclopropyl-(4-hydroxy-phenyl)-methanone has been prepared according to Rastogi, S. N. et al.; J. Med. Chem.; EN; 15; 1972; 286-291.)
Mass Spectrometry: [M + H]⁺ 285
Mass Spectrometry: [M − H]⁺ 283
¹H-NMR (300 MHz, CDCl₃): 0.96-1.03 (m, 2H); 1.16-2.03 (m, 2H); 2.08-2.20 (m, 5H); 2.57-2.66 (m, 2H); 3.76 (t, 2H); 3.98 (t, 2H); 6.90 (d, 2H); 7.61 (s, 1H); 7.95 (d, 2H).
TLC (solvent: DCM/MeOH 95:5, Polygram ALOX) R_{f}: 0,47
MP: 91° C.

| example | structure | Name |
|---|---|---|
| 3/5 | 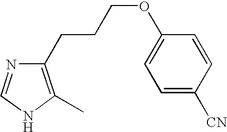 | 4-[3-(5-Methyl-1H- imidazol-4-yl)-propoxy]- benzonitrile |

Example 3/5 has been prepared starting from example 1 and 4-cyanophenol.
Mass Spectrometry: [M + H]⁺ 242
Mass Spectrometry: [M − H]⁻ 239
TLC (solvent: DCM/MeOH = 95:5, Polygram ALOX) R_{f}: 0,48

| example | structure | Name |
|---|---|---|
| 3/6 | 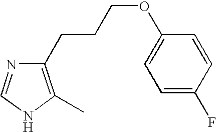 | 4-[3-(4-Fluoro-phenoxy)- propyl]-5-methyl-1H- imidazole |

Example 3/6 has been prepared starting from example 1 and 4-fluorophenol.
Mass Spectrometry: [M + H]⁺ 235
TLC (solvent: DCM/MeOH = 95:5, Polygram ALOX) R_{f}: 0,51
MP: 106° C.

| example | structure | Name |
|---|---|---|
| 3/7 | 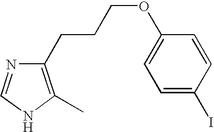 | 4-[3-(4-Iodo-phenoxy)- propyl]-5-methyl-1H- imidazole |

Example 3/7 has been prepared starting from example 1 and 4-iodophenol.
Mass Spectrometry: [M + H]⁺ 343
Mass Spectrometry: [M − H]⁻ 341
TLC (solvent: DCM/MeOH = 95:5 Polygram ALOX), R_{f}: 0,48

| example | structure | Name |
|---|---|---|
| 3/8 | 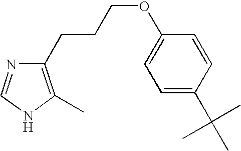 | 4-[3-(4-tert-Butyl- phenoxy)-propyl]-5- methyl-1H-imidazole |

Example 3/8 has been prepared starting from example 1 and 4-tert-butylphenol.
Mass Spectrometry: [M + H]⁺ 273
¹H-NMR (300 MHz, CDCl₃): 1.28 (s, 9H); 2.05-2.13 (m, 2H); 2.16 (s, 3H); 2.70-2.77 (t, 2H); 3.92 (t, 2H); 6.82 (d, 2H); 7.26 (d, 2H); 7.45 (s, 1H).
TLC (solvent: DCM/MeOH = 95:5, Polygram ALOX) R_{f}: 0,53
MP: 97+ C

| Example | structure | Name |
|---|---|---|
| 3/9 | 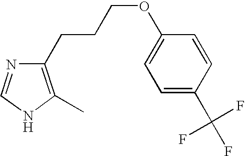 | 5-Methyl-4-[3- (4-trifluoromethyl- phenoxy)-propyl]- 1H-imidazole |

Example 3/9 has been prepared starting from example 1 and 4-(trifluormethyl)phenol.
Mass Spectrometry: [M + ]⁺ 285
TLC (solvent: DCM/MeOH = 95:5, Polygram ALOX) R_{f}: 0,51
MP: 98° C.

| Example | structure | Name |
|---|---|---|
| 3/10 | 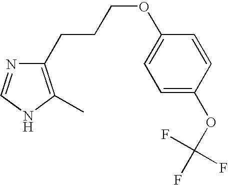 | 5-Methyl-4-[3- (4-trifluoromethoxy- phenoxy)-propyl]- 1H-imidazole |

Example 3/10 has been prepared starting from example 1 and 4-(trifluormethoxy)phenol.
Mass Spectrometry: [M + H]⁺ 301
Mass Spectrometry: [M − H]⁺ 299
TLC (solvent: DCM/MeOH = 95:5, Polygram ALOX) R_{f}: 0,52

| Example | structure | Name |
|---|---|---|
| 3/11 | 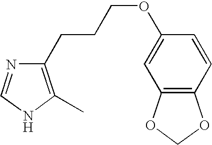 | 4-[3-(Benzo[1,3]dioxol-5-yloxy)-propyl]-5- methyl-1H-imidazole |

Example 3/11 has been prepared starting from example 1 and sesamol.
Mass Spectrometry: [M + H]⁺ 261
Mass Spectrometry: [M − H]⁻ 259
TLC (solvent: DCM/MeOH = 95:5, Polygram ALOX) R_{f}: 0.54

| example | structure | Name |
|---|---|---|
| 3/12 | 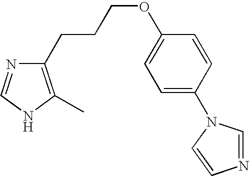 | 4-[3-(4-Imidazol-1-yl-phenoxy)-propyl]-5- methyl-1H-imidazole |

Example 3/12 has been prepared starting from example 1 and 4-(imidazol-1-yl)phenol.
Mass Spectrometry: [M + H]⁺ 283
Mass Spectrometry: [M − H]⁻ 281
¹H-NMR (300 MHz, CDCl₃): 2.11 (p, 2H); 2.17 (s, 3H); 2.74 (t, 2H); 3.97 (t, 2H); 6.90-6.97 (m, 2H); 7.14-7.29 (m, 4H); 7.47 (s, 1H); 7.74 (s, 1H).
TLC (solvent: DCM/MeOH = 95:5, Polygram ALOX) R_{f}: 0.31
MP: 158° C.

| example | structure | Name |
|---|---|---|
| 3/13 | 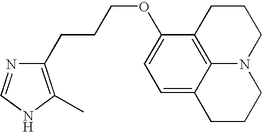 | 8-[3-(5-Methyl-1H-imidazol-4-yl)-propoxy]- 2,3,6,7-tetrahydro-1H,5H-pyrido[3,2,1- ij]quinoline |

Example 3/13 has been prepared starting from example 1 and 8-hydroxyjujolidine.
Mass Spectrometry: [M + H]⁺ 312
Mass Spectrometry: [M − H]⁻ 311
¹H-NMR (300 MHz, CDCl₃): 1.89-2.01 (m, 4H); 2.02-2.12 (m, 2H); 2.17 (s, 3H); 2.64-2.81 (m, 6H); 3.04-3.12 (m, 4H); 3.88 (t, 2H); 6.07 (d, 1H); 6.69 (d, 1H); 7.61 (s, 1H).
TLC (solvent: DCM/MeOH = 95:5, Polygram ALOX) R_{f}: 0.46

| example | structure | Name |
|---|---|---|
| 3/14 | 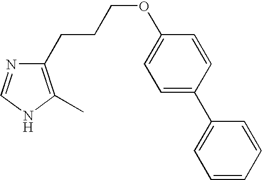 | 4-[3-(Biphenyl-4-yloxy)-propyl]-5-methyl-1H- imidazole |

Example 3/14 has been prepared starting from example 1 and 4-phenylphenol.
Mass Spectrometry: [M + H]⁺ 293
TLC (solvent: DCM/MeOH = 95:5, Polygram ALOX) R_{f}: 0.45
MP: 169° C.

| example | structure | Name |
|---|---|---|
| 3/15 | 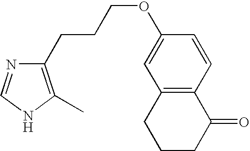 | 6-[3-(5-Methyl-1H-imidazol-4-yl)-propoxy]- 3,4-dihydro-2H-naphthalen-1-one |

Example 3/15 has been prepared starting from example 1 and 6-hydroxy-1-tetralone.
Mass Spectrometry: [M + H]⁺ 285
Mass Spectrometry: [M + H]⁻ 283
TLC (solvent: DCM/MeOH = 95:5, Polygram ALOX) R_{f}: 0.41

| example | structure | Name |
|---|---|---|
| 3/16 | 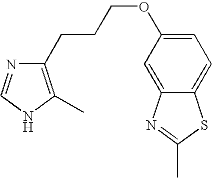 | 2-Methyl-5-[3-(5-methyl-1H-imidazol-4-yl)- propoxy]-benzothiazole |

Example 3/16 has been prepared starting from example 1 and methyl-5-benzothiazolol.
Mass Spectrometry: [M + H]⁺ 288
Mass Spectrometry: [M − H]⁻ 285
TLC (solvent: DCM/MeOH = 95:5, Polygram ALOX) R_{f}: 0.38
MP: 106° C.

| example | structure | Name |
|---|---|---|
| 3/17 | 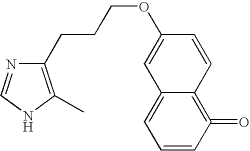 | 5-Methyl-4-[3-(naphthalen-2-yloxy)-propyl]- 1H-imidazole |

Example 3/17 has been prepared starting from example 1 and 2-naphthol.
Mass Spectrometry: [M + H]⁺ 267
TLC (solvent: DCM/MeOH = 95:5, Polygram ALOX) R_{f}: 0.33
MP: 108° C.

| example | structure | Name |
|---|---|---|
| 3/18 | 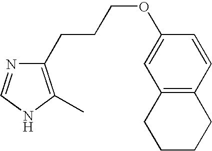 | 5-Methyl-4-[3-(5,6,7,8-tetrahydro-naphthalen- 2-yloxy)-propyl]-1H-imidazole |

Example 3/18 has been prepared starting from example 1 and 5,6,7,8-tetrahydro-2-naphthol.
Mass Spectrometry: [M + H]⁺ 271
Mass Spectrometry: [M − H]⁻ 270
TLC (solvent: DCM/MeOH = 95:5, Polygram ALOX) R_{f}: 0.42

| example | structure | Name |
|---|---|---|
| 3/19 | 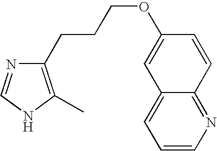 | 6-[3-(5-Methyl-1H-imidazol-4-yl)-propoxy]- quinoline |

Example 3/19 has been prepared starting from example 1 and 6-hydroxyquinoline.
Mass Spectrometry: [M + H]⁺ 268
Mass Spectrometry: [M − H]⁻ 266
TLC (solvent: DCM/MeOH = 95:5, Polygram ALOX) R_{f}: 0.44

| example | structure | Name |
|---|---|---|
| 3/20 | 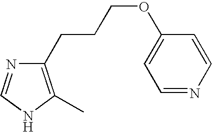 | 4-[3-(5-Methyl-1H-imidazol-4-yl)-propoxy]- pyridine |

Example 3/20 has been prepared starting from example 1 and 4-hydroxypiperidine.
Mass Spectrometry: [M + H]⁺ 218
Mass Spectrometry: [M − H]⁻ 216
TLC (solvent: DCM/MeOH = 95:5, Polygram ALOX) R_{f}: 0.18

| example | structure | Name |
|---|---|---|
| 3/21 | 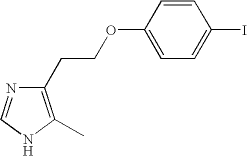 | 2-[3-(5-Methyl-1H- imidazol-4-yl)-propoxy]- pyridine |

Example 3/21 has been prepared starting from example 1 and 2-hydroxypiperidine.
Mass Spectrometry: [M + H]⁺ 218
¹H-NMR (300 MHz, D₆-DMSO): 1.91 (m, 2H); 1.98 (s, 3H); 2.52 (t, 2H); 4.16 (t, 2H); 6.76-6.84 (m, 1H); 6.88-6.97 (m, 1H); 7.35 (s, 1H); 7.61-7.70 (m, 1H); 8.89-8.91 (d, 1H).
TLC (solvent: DCM/MeOH = 95:5, Polygram ALOX) R_{f}: 0,25

### Example 4

### 5-Methyl-4-(4-tert-butyldimethylsilanyloxy-butyl)-1H-imidazole

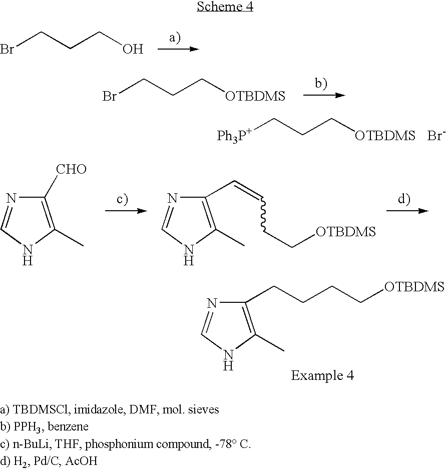

a) To a flame dried flask was added dried powdered molecular sieves (35 g) and 39.2 g (0.575 mol) imidazole and DMF (125 ml). To the stirred suspension was added 3-bromo-1-propanol (20.1 ml, 0.23 mol) and a solution of tert-butyldimethylchlorosilane (41.6 g, 0.276 mol) in DMF (125 ml) at room temperature. The reaction mixture was stirred for 2 hours, filtered and extracted twice with hexane. The hexane phase was washed with water, saturated NaCl-solution, dried (Na₂SO₄) and evaporated. The residue was chromatographed on SiO₂ with hexane to yield the silylated 3-bromo-1-propanol (44.0 g, 75.5%) as a colorless liquid.

b) A mixture of this intermediate (7.7 g, 30.7 mmol) and triphenylphosphine (8.9 g, 33.8 mmol) in benzene was stirred for 72 hours at 80° C. After cooling the reaction mixture was evaporated and the residue chromatographed on SiO₂ with with a gradient of CH₂Cl₂ to CH₂Cl₂/MeOH (20:1) to yield [3-(tert-Butyl-dimethyl-silanyloxy)-propyl]-triphenyl-phosphonium bromide (7.82 g) as a colourless powder. ¹H-NMR (300 MHz, CDCl₃): 7.87-7.65 (m, 15 arom. H); 3.92-3.83 (m, 4H, 2 CH₂); 1.94-1.1.86 (m, 2H, 1 CH₂); 0.85 (s, 9H, tBu); 0.03 (s, 2 CH₃).

c) To a stirred suspension of [3-(tert-butyl-dimethyl-silanyloxy)-propyl]-triphenyl-phosphonium bromide (7.5 g, 14.6 mmol) in THF was added under argon at −78° C. a solution of nBuLi (10.0 ml, 16.0 mmol, 1.6 M in hexane). The orange colored reaction mixture was stirred for 1 hour at −78° C. (which resulted in a clear solution) and 2-methyl-imidazole-3-carboxaldehyde was added in portions. The reaction mixture was stirred for 10 minutes at −78° C., for 20 hours at room temperature and for 8 hours heated under reflux. The reaction mixture was evaporated and the residue chromatographed on SiO₂ with a gradient form EtOAc to a mixture of EtOAc and EtOH (4:1) to yield 5-[-4-(tert-butyl-dimethyl-silanyloxy)-but-1-enyl]-4-methyl-1H-imidazole (1.82 g, 51.9%) as a slightly yellow oil.

d) A mixture of 5-[-4-(tert-Butyl-dimethyl-silanyloxy)-but-1-enyl]-4-methyl-1H-imidazole (1.68 g, 6.3 mmol) and 10% Pd/C (255 mg) in acetic acid was hydrogenated (H₂ balloon) for 15 hours, filtered through celite and the filter residue washed with acetic acid and methanol. The filtrate was evaporated and the residue extracted with EtOAc and 2M aqueous Na₂CO₃ solution. The organic phase was dried (Na₂SO₄) and evaporated to yield 5-Methyl-4-(4-tert-butyl-dimethylsilanyloxy-butyl)-1H-imidazole (1.64 g) as slightly yellow oil.

¹H-NMR (300 MHz, CDCl₃): 7.58 (s, 1H, H—C2); 5.98 (br.s, NH); 3.64 (t, J=6.1, 2H, OCH₂); 2.59 (t, J=7.3, 2H, CH₂—C4); 2.21 (s, 3H, CH₃—C5); 1.72-1.50 (m, 4H, 2 CH₂); 0.89 (s, 9H, tBu); 0.05 (s, 2 CH₃).

### Example 5

### 4-(N-Trityl-5-methyl-1H-imidazol-4-yl)-butanol

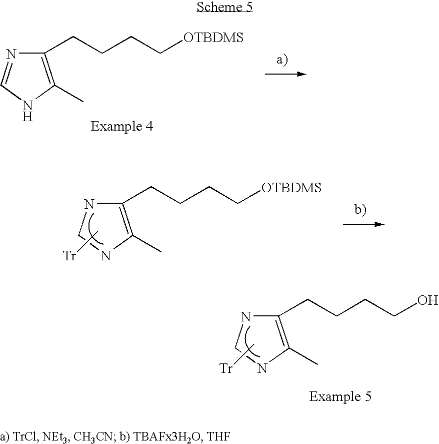

a) To a solution of 5-methyl-4-(4-tert-butyldimethylsilanyloxy-butyl)-1H-imidazole (10 mmol) in acetonitrile (25 ml) were added triethylamine (2.7 ml, 19.4 mmol) and a solution of tritylchloride (4 g, 15 mmol) in acetonitrile (50 ml). The resulting mixture was stirred at 20° C. overnight. The mixture was concentrated under reduced pressure and the residue redissolved in ethylacetate, washed with water (2×) and brine (1×) and concentrated under reduced pressure.

b) To a stirred solution of the tritylated product (2.0 g, 3.9 mmol, as mixture of trityl isomers) in THF (40 ml) was added at room temperature TBAF×3H₂O (1.6 g, 5.1 mmol). After 1.5 and 4.5 hours another two portions of TBAF×3H₂O (800 mg, 2.55 mmol) were added and the reaction mixture was stirred for 5.5 hours and evaporated. The residue was chromatographed on SiO₂ (deactivated with Et₃N) with a gradient of EtOAc/hexane (1:1) to EtOAc yielding example 5 (1.42 g, 91.6%) as colorless powder.

¹H-NMR (300 MHz, DMSO-d6): 7.44-7.36 (m, 9 arom. H); 7.07-6.97 (m, 6 arom. H, H—C2); 4.32 (t, J=5.2, 1H, OH); 3.36 (br.qa, J=5.2, 6.2; 1H, CH₂O); 2.35 (t, J=7.2, 2H, CH₂—C4); 1.58-1.32 (m, 4H, 2 CH₂); 1.30 (s, 3H, CH₃—C5).

### Example 6

### 5-Methyl-4-(2-tert-butyldimethylsilanyloxy-ethyl)-1H-imidazole

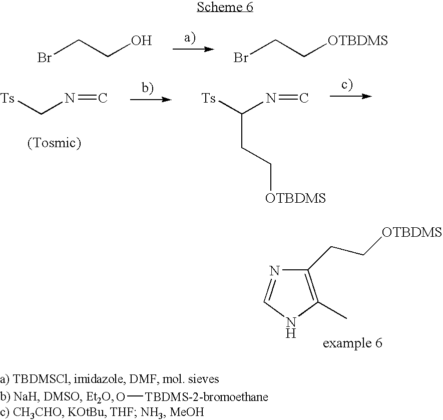

a) The TBDMS protected alcohol was prepared from 2-bromo-ethanol using the procedure described in 4 a).

b) In a dried flask NaH-dispersion (7.4 g, ˜0.170 mol) was washed twice with hexane and suspended in Et₂O (90 ml) and DMSO (270 ml) and cooled to 4° C. To the reaction mixture was added a solution of Tosmic (30 g, 0.153 mol) in a mixture of DMSO (180 ml) and Et₂O (60 ml) and stirred for 5 minutes, followed by addition of a solution of the silylated 2-bromo-ethanol (42.8 g, 0.169 mol) in Et₂O (150 ml) whereby the temperature was held between 10-15° C. The reaction mixture was stirred for 45 minutes at room temperature, poured onto cooled water (750 ml) and extracted twice with EtOAc (1 1). The combined organic fractions were washed with water and saturated brine, dried (Na₂SO₄), evaporated and the residue chromatographed on SiO₂ with a gradient of hexane to hexane/EtOAc 4:1) to yield [3-TBDMS-1-(toluene-4-sulfonyl)-propyl]-isocyanide (35.0 g, 64.1%) as a clear oil. ¹H-NMR (300 MHz, CDCl₃): 7.87 (d, J=8.2, 2 arom. H); 7.42 (d, J=8.1, 2 arom. H); 4.76 (dd, J=3.3, 11.0, 1H); 3.92-3.71 (m, 2H, OCH₂); 2.49 (s, 3H, CH₃); 2.49-2.40 (m, 1H); 1.94-1.83 (m, 1H); 0.88 (s, 9H, tBu); 0.06 (s, 6H, 2 CH₃).

c) To [4-TBDMS-1-(toluene-4-sulfonyl)-butyl]-isocyanid (7.0 mmol) was added THF (25 ml), acetaldehyde (7.7 mmol) and KOtBu (250 mg) at room temperature (reaction slightly exothermic). The reaction mixture was stirred for 20 minutes at room temperature, the solvent was removed and the residue dissolved in saturated methanolic ammonia (25 ml) and heated at 100° for 5 hours in a sealed tube. After cooling to room temperature the solvent was removed and the residue chromatographed on SiO₂ with a gradient from EtOAc to EtOAc and ethanol (7:3) yielding example 6 (33%) as a slighly yellow oil.

¹H-NMR (300 MHz, CDCl₃): 7.57 (s, 1H, H—C2); 6.14 (br.s, NH); 3.83 (t, J=6.0, 2H, OCH₂); 2.76 (t, J=6.0, 2H, CH₂—C4); 2.20 (s, 3H, CH₃—C5); 0.88 (s, 9H, 6H, 2 CH₃).

### Example 7

### 2-(N-Trityl-5-methyl-1H-imidazol-4-yl)-ethanol

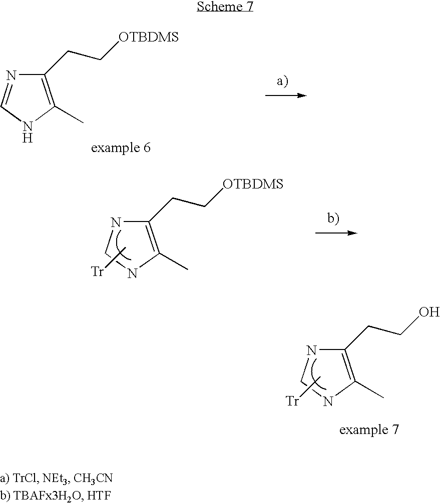

a) To a solution of 5-methyl-4-(2-tert-butyldimethylsilanyloxy-ethyl)-1H-imidazole (30 g, 125 mmol) in acetonitrile (300 ml) were added triethylamine (34 ml, 250 mmol) and a solution of tritylchloride (52.3 g, 188 mmol) in acetonitrile (600 ml). The resulting mixture was stirred at 20° C. overnight. The mixture was concentrated under reduced pressure and the residue redissolved in ethylacetate, washed with water (2×) and brine (1×) and concentrated under reduced pressure.

b) To a stirred solution of the tritylated product (2.0 g, 3.9 mmol, as mixture of trityl isomers) in THF (40 ml) was added at room temperature TBAF×3H₂O (1.6 g, 5.1 mmol). After 1.5 and 4.5 hours another two portions of TBAF×3H₂O (800 mg, 2.55 mmol) were added and the reaction mixture was stirred for 5.5 hours and evaporated. The residue was chromatographed on SiO₂ (deactivated with Et₃N) with a gradient of EtOAc/hexane (1:1) to EtOAc yielding example 7 (1.42 g, 91.6%) as colorless powder.

### Example 8

### 5-Ethyl-4-(3-tert-butyldimethylsilanyloxy-propyl)-1H-imidazole

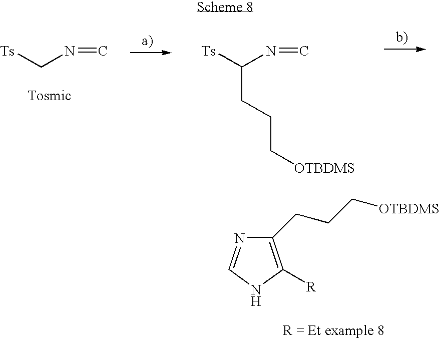

a) In a first step tosmic was alkylated with the silylated 3-bromo-1-propanol, described in the preparation of example 4, using the same reaction conditions as described for the respective precursor of example 6.

¹H-NMR (300 MHz, CDCl₃): 7.86 (d, J˜8.1, 2 arom. H); 7.42 (d, J˜7.9, 2 arom. H); 4.69 (dd, J=3.4, 10.5, H—C1); 3.75-3.60 (m, 2H, OCH₂); 2.49 (s, 3H, CH₃); 2.37-2.26 (m, 1H); 1.98-1.67 (m, 3H); 0.86 (m, 9H, tBu); 0.04 (m, 6H, 2 CH₃).

b) To [4-TBDMS-1-(toluene-4-sulfonyl)-butyl]-isocyanid (2.5 g, 7.0 mmol) was added THF (25 ml), propionaldehyde (450 mg, 7.7 mmol) and KOtBu (250 mg) at room temperature (reaction slightly exothermic). The reaction mixture was stirred for 20 minutes at room temperature, the solvent was removed and the residue dissolved in saturated methanolic ammonia (25 ml) and heated at 100° for 5 hours in a sealed tube. After cooling to room temperature the solvent was removed and the residue chromatographed on SiO₂ with a gradient from EtOAc to EtOAc and Ethanol (7:3) yielding example 8 (550 mg, 29.1%) as a slighly yellow oil.

¹H-NMR (300 MHz, CDCl₃): 7.53 (s, 1H, H—C2); ˜6.6 (br.s, NH); 3.65 (t, J=˜6.0, 2H, OCH₂); 2.66 (t, J=7.1, 2H, CH₂—C4); 2.57 (qa, J=7.5, 2H, CH₂—C5); 1.81 (m, 2H, CH₂); 1.21 (t, J=7.5, 3H, CH₃); 0.90 (s, 9H, tBu); 0.06 (s, 6H, 2 CH₃).

### Example 9

### 2-(N-Trityl-5-ethyl-1H-imidazol-4-yl)-propanol

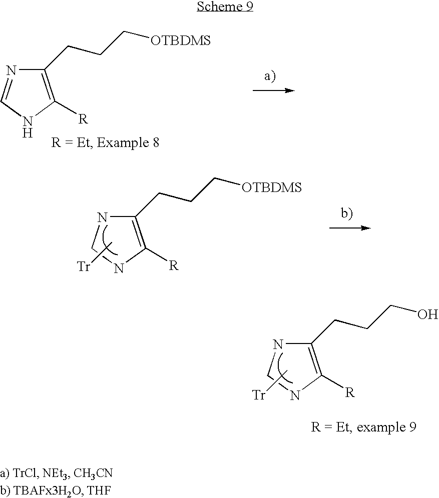

a) To a solution of 5-Ethyl-4-(3-tert-butyldimethylsilanyloxy-propyl)-1H-imidazole (1,96 mmol, 500 mg) in acetonitrile (5 ml) were added triethylamine (550 μl, 3.9 mmol) and a solution of tritylchloride (820 mg, 3 mmol) in acetonitrile (10 ml). The resulting mixture was stirred at 20° C. overnight. The mixture was concentrated under reduced pressure and the residue redissolved in ethylacetate, washed with water (2×) and brine (1×) and concentrated under reduced pressure.

b) To a stirred solution of the tritylated product in THF (20 ml) was added at room temperature TBAF×3H₂O (800 mg, 2.5 mmol). After 1.5 and 4.5 hours another two portions of TBAF×3H₂O (400 mg, 1.3 mmol) were added and the reaction mixture was stirred for 5.5 hours and evaporated. The residue was chromatographed on SiO₂ (deactivated with Et₃N) with a gradient of EtOAc/hexane (1:1) to EtOAc yielding 2-(N-trityl-5-ethyl-1H-imidazol4-yl)-propanol (653 mg, 87%) as colorless powder.

¹H-NMR (300 MHz, DMSO-d6, ratio of isomers ˜1:1): 7.41-7.30 (m, 2×9 arom. H); 7.07-7.05 (m, 2×6 arom. H); 6.97 and 6.96 (2s, 2H, H—C2, two isomers); 4.45 and 4.11 (2t, J=5.0 and 4.9, 2×1H, OH, two isomers); 3.41 and 2.75 (2td, ˜qa, J˜6.2, 5.1 and 5.9, 5.1, 2×2H, OCH₂, two isomers); 2.42-2.34 and 2.06-1.98 (2m, 2×4H, CH₂); 1.72 (m, 2H, CH₂); 1.12 (t, J=7.5, 3H, CH₃); 0.44-0.37 (m, 2H, CH₂); 0.02 (t, J=7.3, 3H, CH₃).

### Example 10

### 5-Isopropyl-4-(3-tert-butyldimethylsilanyloxy-propyl)-1H-imidazole

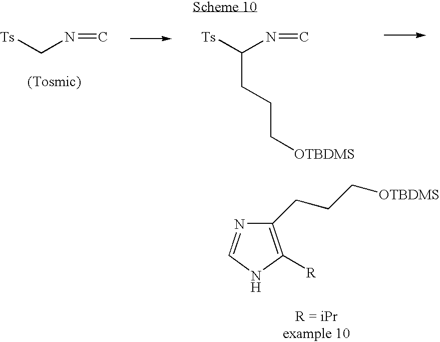

5-Isopropyl-4-(3-tert-butyldimethylsilanyloxy-propyl)-1H-imidazole was prepared using the procedure described for example 8a. The subsequent transformation to the imidazole was carried out according to example 8b using isobutyraldehyde.

¹H-NMR (300 MHz, CDCl₃): 7.62 (s, 1H, H—C2); ˜6.5 (br.s, NH); 3.64 (t, J=5.9, 2H, OCH₂); 3.00 (sp, J˜6.9, 1H, CH—C5); 2.67 (t, J=7.2, 2H, CH₂—C4); 1.81 (m, 2H, CH₂); 1.26 (d, J=7.0, 6H, 2 CH₃); 0.90 (s, 9H, tBu); 0.06 (s, 6H, 2 CH₃).

### Example 11

### 2-(N-Trityl-5-isopropyl-1H-imidazol-4-yl)-propanol

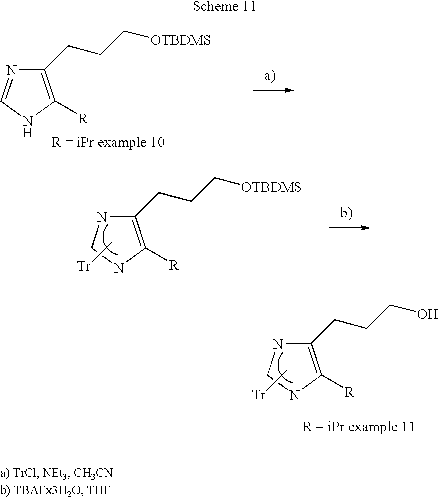

Tritylation of the imidazole ring and removal of the TBDMS protection group has been carried out using the procedure described in example 9.

¹H-NMR (300 MHz, DMSO-d6): 7.41-7.29 (m, 9 arom. H); 7.07-7.04 (m, 6 arom. H); 6.96 (s, H—C2); 4.10 (t, J=4.9, 1H, OH); 2.78-2.71 (m, 3H, OCH₂, CH—C5); 2.06-1.97 (m, 2H, CH₂); 1.13 (d, J=6.8, 6H, CH₃); 0.40-0.34 (m, 2H, CH₂).

### Example 12

### 5-Phenyl-4-(3-tert-butyldimethylsilanyloxy-propyl)-1H-imidazole

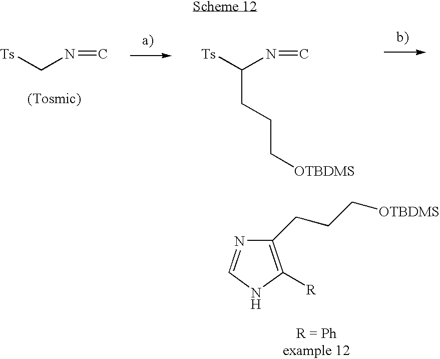

a) Preparation of the proyplated Tosmic intermediate has been carried out according to example 8.

b) To a stirred solution of this intermediate (4.54 g, 12.8 mmol) in saturated methanolic ammonia (50 ml) was added benzaldehyde (1.41 ml, 14.1 ml) at room temperature. After stirring for 10 minutes at room temperature the reaction mixture was heated for 15 hours at 100° C., cooled to room temperature and evaporated. The residue was chromatographed on SiO₂ with a gradient of CHCl₃ to CHCl₃/EtOH (9:1) yielding example 12 (1.78 g, 44%) as a slightly yellow oil.

¹H-NMR (300 MHz, CDCl₃): 7.75 (s, 1H, H—C5); 7.61-7.29 (m, 5 arom. H, NH); 3.72 (t, J=5.7, 2H, OCH₂); 2.95 (t, J=7.1, 2H, CH₂—C4); 1.93 (tt, 2H, CH₂); 0.92 (s, 9H, tBu); 0.08 (s, 6H, 2 CH₃).

### Example 13

### 2-(N-Trityl-5-phenyl-1H-imidazol-4-yl)-propanol

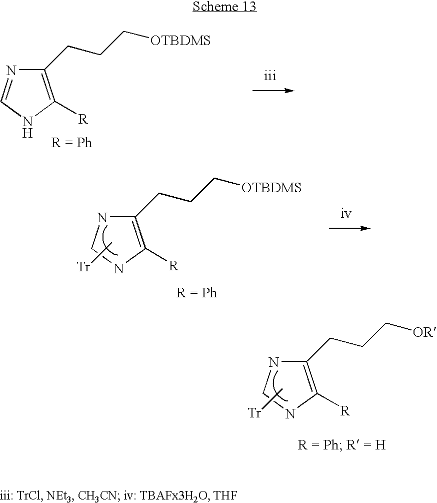

Tritylation of the imidazole ring and removal of the TBDMS protection group has been carried out using the procedure described in example 9.

¹H-NMR (300 MHz, DMSO-d6): 7.75-7.72 (m, 2 arom. H); 7.46-7.31 (m, 11 arom. H); 7.22-7.15 (m, 8 arom. H); 4.14 (t, J=4.9, 1H, OH); 2.73 (dt, ˜qa, J˜6.0, 2H, OCH₂); 2.42 (m, 2H, CH₂); 0.39 (m, 2H, CH₂—C4).

### Examples 14/1-14/6

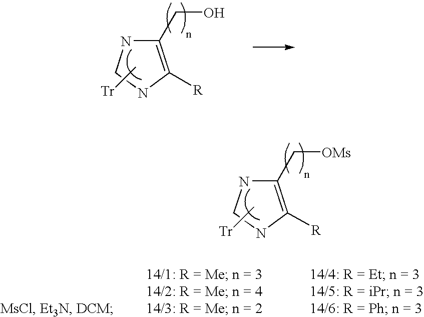

The following general procedure was applied for the mesylation of examples 1, 5, 7, 9, 11, 13:

To a stirred solution of one of the alcohols 1, 5, 7, 9, 11 or 13 (10.8 mmol) in CH₂Cl₂ (40 ml) was added at 4° C. Et₃N (2.72 g, 26.9 mmol) and slowly a solution of methanesufonylchloride (1.08 ml, 14.0 mmol) in CH₂Cl₂ (10 ml). The reaction mixture was stirred for 1 hour at 4° C. and poured onto H₂O (100 ml) and EtOAc (150 ml). The organic phase was extracted with saturated NaHCO₃-solution and saturated brine, dried (Na₂SO₄), the solvents were evaporated and the residue dried at high vacuum yielding the corresponding mesylates usually as amorphous solids which were not further purified.

### Examples 15/1-15/13

### Standard Procedure for the Arylether Products

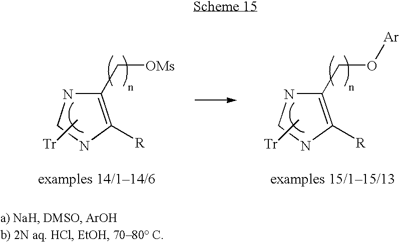

To a stirred solution of an appropriate phenol (1.3 mmol) in DMSO (2 ml) a NaH-dispersion (1.5 mmol) was added at room temperature (H₂ evolution). The reaction mixture was stirred for 30 minutes at room temperature followed by addition of mesylates 14/1-14/6 (1.0 mmol) in DMSO (1 ml) and stirring at 70° C. for 1-3 hours. After addition of H₂O and EtOAc the organic layer was dried (MgSO₄) and the solvents were evaporated. The residue was chromatographed using gradients of EtOAc and hexane and the product dissolved in EtOH (2 ml) and 2N aqueous HCl-solution (4 ml) was added followed by heating the reaction mixture for 2-4 hours (TrOH usually precipitates during the course of the reaction). After cooling to room temperature the suspension was filtered, the filtrate evaporated to dryness and the residue extracted with 10% aqueous Na₂CO₃-solution and CHCl₃. The organic fraction was evaporated and the residue chromatographed on SiO₂ using a gradient of CHCl₃ to CHCl₃/MeOH (5:1) yielding the products usually as amorphous solids.

| example | structure | Name |
|---|---|---|
| 15/1 | 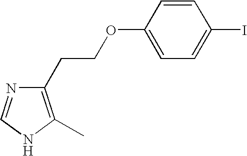 | 4-[2-(4-Iodo- phenoxy)-ethyl]-5- methyl-1H- imidazole |

Example 15/1 has been prepared starting from example 14/3 and 4-iodophenol.
Mass Spectrometry: [M + H]⁺ 329
Mass Spectrometry: [M − H]⁻ 327
TLC (solvent: DCM/MeOH = 95:5, Polygram ALOX) R_{f}: 0,37
MP: 160° C.

| example | structure | Name |
|---|---|---|
| 15/2 | 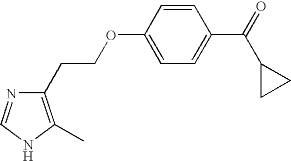 | Cyclopropyl- {4-[2-(5- methyl-1H- imidazol-4- yl)-ethoxy]- phenyl}- methanone |

Example 15/2 has been prepared starting from example 14/3 and cyclopropyl-(4-hydroxy-phenyl)-methanone.
Mass Spectrometry: [M + H]⁺ 271
TLC (solvent: DCM/MeOH = 95:5, Polygram ALOX) R_{f}: 0,34
MP: 163° C.

| example | structure | Name |
|---|---|---|
| 15/3 | 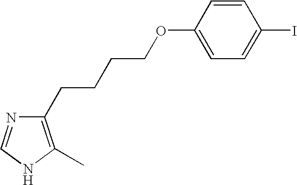 | 4-[4-(4-Iodo- phenoxy)- butyl]-5- methyl-1H- imidazole |

Example 15/3 has been prepared starting from example 14/2 and 4-iodophenol.
Mass Spectrometry: [M + H]⁺ 357
TLC (solvent: DCM/MeOH = 95:5, Polygram ALOX) R_{f}: 0,46
MP: 125° C.

| example | structure | Name |
|---|---|---|
| 15/4 | 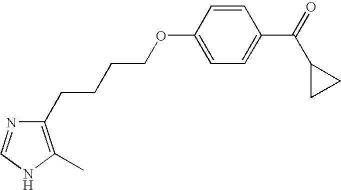 | Cyclopropyl-{4-[4-(5-methyl-1H-imidazol-4- yl)-butoxy]-phenyl}-methanone |

Example 15/4 has been prepared starting from example 14/2 and cyclopropyl-(4-hydroxy-phenyl)-methanone.
Mass Spectrometry: [M + H]⁺ 299
Mass Spectrometry: [M − H]⁻ 297
TLC (solvent: DCM/MeOH = 95:5, Polygram ALOX) R_{f}: 0,34

| example | structure | Name |
|---|---|---|
| 15/5 | 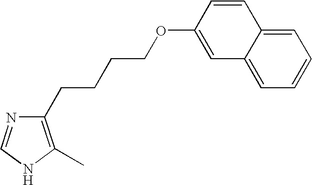 | 5-Methyl-4-[4-(naphthalen-2-yloxy)-butyl]-1H- imidazole |

Example 15/5 has been prepared starting from example 14/2 and 2-naphthol.
Mass Spectrometry: [M + H]⁺ 281
Mass Spectrometry: [M − H]⁻ 279
TLC (solvent: DCM/MeOH = 95:5, Polygram ALOX) R_{f}: 0,41
MP: 85° C.

| example | structure | Name |
|---|---|---|
| 15/6 | 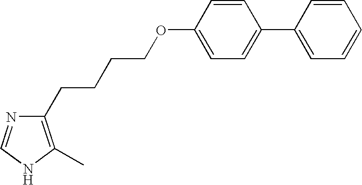 | 4-[4-(Biphenyl-4-yloxy)-butyl]-5-methyl-1H- imidazole |

Example 15/6 has been prepared starting from example 14/2 and 4-phenylphenol.
Mass Spectrometry: [M + H]⁺ 308
Mass Spectrometry: [M − H]⁻ 306
TLC (solvent: DCM/MeOH = 95:5, Polygram ALOX) R_{f}: 0,39
MP: 136° C.

| example | structure | Name |
|---|---|---|
| 15/7 | 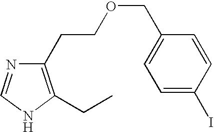 | 5-Ethyl-4-[3-(4-iodo- phenoxy)-propyl]-1H- imidazole |

Example 15/7 has been prepared starting from example 14/4 and 4-iodophenol.
Mass Spectrometry: [M + H]⁺ 357
Mass Spectrometry: [M − H]⁻ 356
TLC (solvent: DCM/MeOH = 95:5, Polygram ALOX) R_{f}: 0,56
MP: 91° C.

| example | structure | Name |
|---|---|---|
| 15/8 | 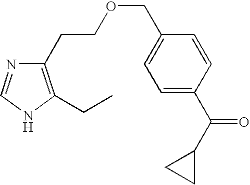 | Cyclopropyl-{4-[3- (5-ethyl-1H- imidazol-4-yl)- propoxy]-phenyl}- methanone |

Example 15/8 has been prepared starting from example 14/4 and cyclopropyl-(4-hydroxy-phenyl)-methanone.
Mass Spectrometry: [M + H]⁺ 300
Mass Spectrometry: [M − H]⁻ 298
TLC (solvent: DCM/MeOH = 95:5, Polygram ALOX) R_{f}: 0,56
MP: 115° C.

| example | structure | Name |
|---|---|---|
| 15/9 | 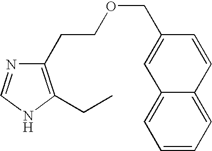 | 5-Ethyl-4-[3-(naphthalen- 2-yloxy)-propyl]-1H- imidazole |

Example 15/9 has been prepared starting from example 14/4 and 2-naphthol.
Mass Spectrometry: [M + H]⁺ 281
Mass Spectrometry: [M − H]⁻ 280
TLC (solvent: DCM/MeOH = 95:5, Polygram ALOX) R_{f}: 0,57
MP: 92° C.

| ex- | | |
|---|---|---|
| am- | | |
| ple | Structure | Name |
| 15/10 | 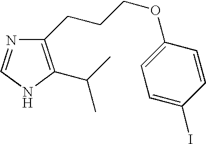 | 4-[3-(4-Iodo- phenoxy)-propyl]-5-iso- propyl-1H-imidazole |

Example 15/10 has been prepared starting from example 14/5 and 4-iodophenol.
Mass Spectrometry: [M + H]⁺ 371
Mass Spectrometry: [M − H]⁻ 369
TLC (solvent: DCM/MeOH = 95:5, Polygram ALOX) R_{f}: 0,56
MP: 111° C.

| ex- | | |
|---|---|---|
| am- | | |
| ple | Structure | Name |
| 15/11 | 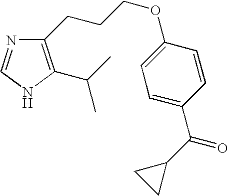 | Cyclopropyl-{4-[3-(5-iso- propyl-1H-imida- zol-4-yl)-pro- poxy]-phenyl}-methanone |

Example 15/11 has been prepared starting from example 14/5 and cyclopropyl-(4-hydroxy-phenyl)-methanone.
Mass Spectrometry: [M + H]⁺ 313
Mass Spectrometry: [M − H]⁻ 311
TLC (solvent: DCM/MeOH = 95:5, Polygram ALOX) R_{f}: 0,55
MP: 143° C.

| ex- | | |
|---|---|---|
| am- | | |
| ple | Structure | Name |
| 15/12 | 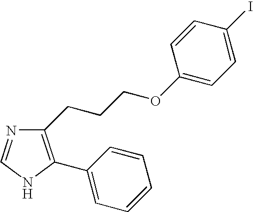 | 4-[3-(4-Iodo- phenoxy)-pro- pyl]-5-phenyl-1H-imi- dazole |

Example 15/12 has been prepared starting from example 14/6 and 4-iodophenol.
Mass Spectrometry: [M + H]⁺ 405
Mass Spectrometry: [M − H]⁻ 403
MP: 118° C.

| exam- | | |
|---|---|---|
| ple | structure | Name |
| 15/13 | 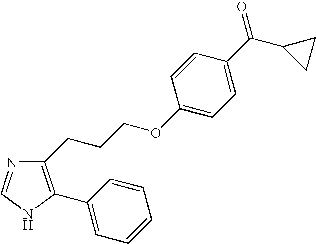 | Cyclopropyl-{4-[3-(5-phenyl-1H-imidazol-4-yl)-pro- poxy]-phenyl}-methanone |

Example 15/13 has been prepared starting from example 14/6 and cyclopropyl-(4-hydroxy-phenyl)-methanone.
Mass Spectrometry: [M + H]⁺ 347
Mass Spectrometry: [M − H]⁻ 346
TLC (solvent: DCM:MeOH = 95:5, Polygram ALOX) R_{f}: 0,50
MP: 154° C.

### Examples 16/1-16/47

### Standard Procedure for the Benzylether Products

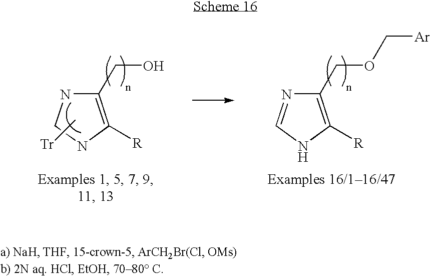

To a stirred solution of one of the alcohols 1, 5, 7, 9, 11 or 13 (1.0 mmol) in THF (3 ml) was added NaH-dispersion (2.0 mmol) and 15-crown-5 (10 μl) at room temperature (H₂ evolution). The reaction mixture was stirred for 30 minutes and cooled to 4° C. followed by addition of the appropriate benzylhalide(1.1 mmol) and stirring at room temperature (gentle heating if required) for 1-24 hours. After addition of H₂O and EtOAc the organic layer was dried (MgSO₄) and the solvents were evaporated.

The residue was chromatographed using gradients of EtOAc and hexane and the product dissolved in EtOH (2 ml) and 2N aqueous HCl-solution (4 ml) was added followed by heating the reaction mixture for 2-4 hours (TrOH usually precipitates during the course of the reaction). After cooling to room temperature the suspension was filtered, the filtrate extracted with 10% aqueous Na₂CO₃-solution and CHCl₃. The organic fraction was evaporated and the residue chromatographed on SiO₂ using a gradient of CHCl₃ to CHCl₃/MeOH (5:1) yielding the products usually as amorphous solids.

| exam- | | |
|---|---|---|
| ple | Structure | Name |
| 16/1 | 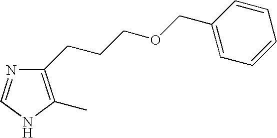 | 4-(3-Benzyloxy-propyl)-5-methyl-1H-imi- dazole |

Example 16/1 has been prepared starting from example 1 and benzylbromide.
Mass Spectrometry: [M + H]⁺ 231
Mass Spectrometry: [M − H]⁻ 229
¹H-NMR(300 MHz, CDCl₃): 1.88(p, 2H); 2.15(s, 3H); 2.64(t, 2H); 3.51(t, 2H); 4.50(s, 2H); 7.24-7.39(m, 6H).
TLC(solvent: DCM/MeOH = 95:5, Polygram ALOX) R_{f}: 0,38

| exam- | | |
|---|---|---|
| ple | Structure | Name |
| 16/2 | 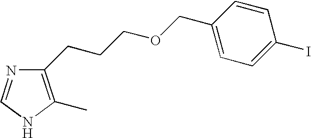 | 4-[3-(4-Iodo-benzyloxy)-propyl]-5-methyl-1H-imi- dazole |

Example 16/2 has been prepared starting from example 1 and 4-iodobenzylbromide.
Mass Spectrometry: [M + H]⁺ 357
Mass Spectrometry: [M − H]⁻ 355
TLC (solvent: DCM/MeOH = 95:5, Polygram ALOX) R_{f}: 0,37

| exam- | | |
|---|---|---|
| ple | structure | Name |
| 16/3 | 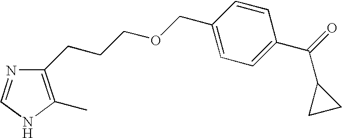 | Cyclopropyl-{4-[3-(5-methyl-1H-imidazol-4-yl)-propoxy- methyl]-phenyl}-methanone |

Example 16/3 has been prepared starting from example 1 and p-cyclopropyl-carbonyl-benzylbromid.
Mass Spectrometry: [M + H]⁺ 300
TLC (solvent: DCM/MeOH = 95:5, Polygram ALOX) R_{f}: 0,35

| exam- | | |
|---|---|---|
| ple | structure | Name |
| 16/4 | 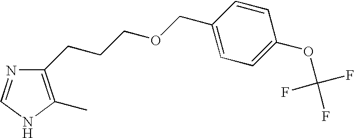 | 5-Methyl-4-[3-(4-trifluoromethoxy-benzyl- oxy)-propyl]-1H-imidazole |

Example 16/4 has been prepared starting from example 1 and 4-(trifluormethoxy)-benzylbromid.
Mass Spectrometry: [M − H]⁻ 313
TLC (solvent: DCM/MeOH = 95:5, Polygram ALOX) R_{f}: 0,41

| exam- | | |
|---|---|---|
| ple | Structure | Name |
| 16/5 | 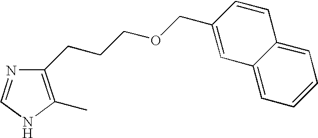 | 5-Methyl-4-[3-(naphthalen-2-ylmethoxy)-pro- pyl]-1H-imidazole |

Example 16/5 has been prepared starting from example 1 and 2-(bromomethyl)-naphthalene.
Mass Spectrometry: [M + H]⁺ 281
Mass Spectrometry: [M − H]⁻ 280
TLC (solvent: DCM/MeOH = 95:5, Polygram ALOX) R_{f}: 0,55

| exam- | | |
|---|---|---|
| ple | structure | Name |
| 16/6 | 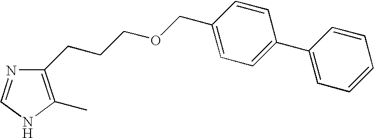 | 4-[3-(Biphenyl-4-ylmethoxy)-propyl]-5-meth- yl-1H-imidazole |

Example 16/6 has been prepared starting from example 1 and 4-bromomethylbiphenyl.
Mass Spectrometry: [M + H]⁺ 307
Mass Spectrometry: [M − H]⁻ 305
TLC (solvent: DCM/MeOH = 95:5, Polygram ALOX) R_{f}: 0.58
MP: 95° C.

| example | structure | Name |
|---|---|---|
| 16/7 | 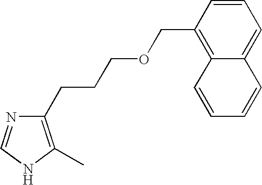 | 5-Methyl-4-[3-(naphthalen-1-ylmethoxy)- propyl]-1H-imidazole |

Example 16/7 has been prepared starting from example 1 and 1-(chloromethyl)-naphthalene.
Mass Spectrometry: [M + H]⁺ 281
Mass Spectrometry: [M − H]⁻ 280
TLC (solvent: DCM/MeOH = 95:5, Polygram ALOX) R_{f}: 0.54

| example | structure | Name |
|---|---|---|
| 16/8 | 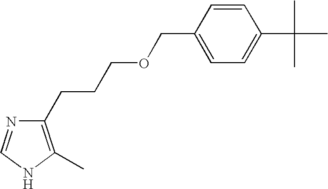 | 4-[3-(4-tert-Butyl-benzyloxy)-propyl]-5- methyl-1H-imidazole |

Example 16/8 has been prepared starting from example 1 and 4-(tert-butyl)benzylbromide.
Mass Spectrometry: [M + H]⁺ 288
¹H-NMR (300 MHz, CDCl₃): 1.32 (s, 9H); 1.88 (p, 2H); 2.15 (s, 3H); 2.65 (t, 2H); 3.50 (t, 2H); 4.49 (s, 2H); 7.22-7.44 (m, 3H).
TLC (solvent: DCM/MeOH = 95:5, Polygram ALOX) R_{f}: 0.48

| example | structure | Name |
|---|---|---|
| 16/9 | 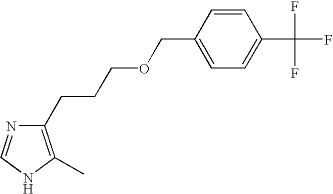 | 5-Methyl-4-[3-(4-trifluoromethyl-benzyloxy)- propyl]-1H-imidazole |

Example 16/9 has been prepared starting from example 1 and 4-(trifluoromethyl)-benzylbromide.
Mass Spectrometry: [M + H]⁺ 299
Mass Spectrometry: [M − H]⁻ 297
¹H-NMR (300 MHz, CDCl₃): 1.92 (p, 2H); 2.20 (s, 3H); 2.67 (t, 2H); 3.53 (t, 2H); 4.47 (s, 2H); 7.41-7.45 (m, 3H); 7.60 (d, 2H).
TLC (solvent: DCM/MeOH = 95:5, Polygram ALOX) R_{f}: 0.48

| example | structure | Name |
|---|---|---|
| 16/10 | 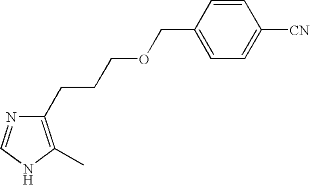 | 4-[3-(5-Methyl-1H-imidazol-4-yl)- propoxymethyl]-benzonitrile |

Example 16/10 has been prepared starting from example 1 and 4-cyanobenzylbromide.
Mass Spectrometry: [M + H]⁺ 256
Mass Spectrometry: [M − H]⁻ 254
TLC (solvent: DCM/MeOH = 95:5, Polygram ALOX) R_{f}: 0.46

| example | structure | Name |
|---|---|---|
| 16/11 | 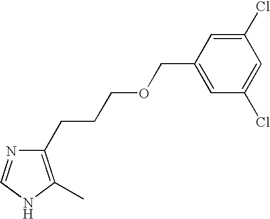 | 4-[3-(3,5-Dichloro-benzyloxy)-propyl]-5- methyl-1H-imidazole |

Example 16/11 has been prepared starting from example 1 and 3,5-dichlorobenzyl-choride.
Mass Spectrometry: [M + H]⁺ 299
Mass Spectrometry: [M − H]⁻ 297
¹H-NMR (300 MHz, CDCl₃): 1.92 (p, 2H); 2.18 (s, 3H); 2.64 (t, 2H); 3.50 (t, 2H); 4.43 (s, 2H); 7.20 (s, 2H); 7.26 (s, 1H); 7.44 (s, 1H).
TLC (solvent: DCM/MeOH = 95:5, Polygram ALOX) R_{f}: 0.52

| example | structure | Name |
|---|---|---|
| 16/12 | 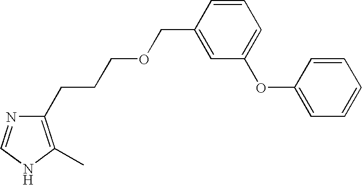 | 5-Methyl-4-[3-(3-phenoxy-benzyloxy)- propyl]-1H-imidazole |

Example 16/12 has been prepared starting from example 1 and 3-phenoxy-benzyl-chloride.
Mass Spectrometry: [M + H]⁺ 323
Mass Spectrometry: [M − H]⁻ 322
TLC (solvent: DCM/MeOH = 95:5, Polygram ALOX) R_{f}: 0.54

| example | structure | Name |
|---|---|---|
| 16/13 | 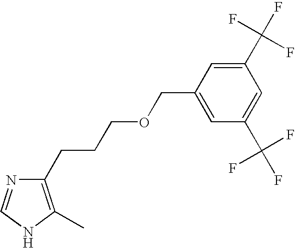 | 4-[3-(3,5-Bis-trifluoromethyl-benzyloxy)- propyl]-5-methyl-1H-imidazole |

Example 16/13 has been prepared starting from example 1 and 3,5-bis(trifluoromethyl)-benzylbromide.
Mass Spectrometry: [M + H]⁺ 367
Mass Spectrometry: [M − H]⁻ 365
TLC (solvent: DCM/MeOH = 95:5, Polygram ALOX) R_{f}: 0.62
MP: 90° C.

| example | structure | Name |
|---|---|---|
| 16/14 | 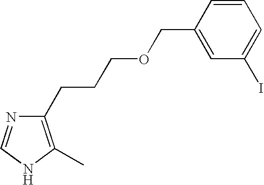 | 4-[3-(3-Iodo-benzyloxy)-propyl]-5-methyl- 1H-imidazole |

Example 16/14 has been prepared starting from example 1 and 3-iodobenzylbromide.
Mass Spectrometry: [M + H]⁺ 357
¹H-NMR (300 MHz, CDCl₃): 1.91 (p, 2H); 2.16 (s, 3H); 2.65 (t, 2H); 3.50 (t, 2H); 4.43 (s, 2H); 7.08 (t, 1H); 7.22-7.43 (m, 2H); 7.62 (d, 1H); 7.69 (s, 1H).
TLC (solvent: DCM/MeOH = 95:5, Polygram ALOX) R_{f}: 0.61

| example | Structure | Name |
|---|---|---|
| 16/15 | 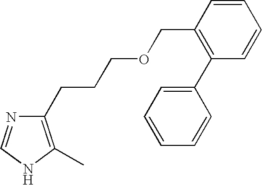 | 4-[3-(Biphenyl-2-ylmethoxy)-propyl]-5- methyl-1H-imidazole |

Example 16/15 has been prepared starting from example 1 and 2-phenylbenzylbromide.
Mass Spectrometry: [M + H]⁺ 307
Mass Spectrometry: [M − H]⁻ 305
TLC (solvent: DCM/MeOH = 95:5, Polygram ALOX) R_{f}: 0.61

| example | Structure | Name |
|---|---|---|
| 16/16 | 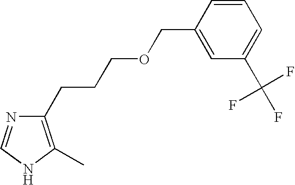 | 5-Methyl-4-[3-(3-trifluoromethyl-benzyloxy)- propyl]-1H-imidazole |

Example 16/16 has been prepared starting from example 1 and 3-(trifluoromethyl)-benzylbromide.
Mass Spectrometry: [M + H]⁺ 299
Mass Spectrometry: [M − H]⁻ 297

| example | structure | Name |
|---|---|---|
| 16/17 | 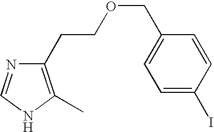 | 4-[2-(4-Iodo-benzyloxy)- ethyl]-5-methyl-1H- imidazole |

Example 16/17 has been prepared starting from example 7 and 4-iodobenzylbromide.
Mass Spectrometry: [M + H]⁺ 343
Mass Spectrometry: [M − H]⁻ 341
TLC (solvent: DCM/MeOH = 95:5, Polygram ALOX) R_{f}: 0,64
MP: 74° C.

| example | structure | Name |
|---|---|---|
| 16/18 | 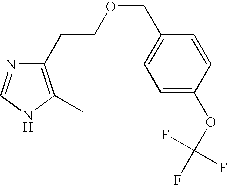 | 5-Methyl-4-[2- (4-trifluoromethoxy- benzyloxy)-ethyl]-1H- imidazole |

Example 16/18 has been prepared starting from example 7 and 4-(trifluormethoxy)-benzylbromide.
Mass Spectrometry: [M + H]⁺ 301
Mass Spectrometry: [M − H]⁻ 300
TLC (solvent: DCM/MeOH = 95:5, Polygram ALOX) R_{f}: 0,65
MP: 66° C.

| example | structure | Name |
|---|---|---|
| 16/19 | 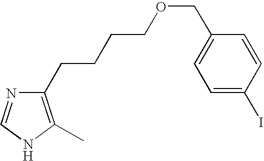 | 4-[4-(4-Iodo- benzyloxy)- butyl]-5-methyl- 1H-imidazole |

Example 16/19 has been prepared starting from example 5 and and 4-iodobenzylbromide.
Mass Spectrometry: [M + H]⁺ 371
Mass Spectrometry: [M − H]⁻ 369
TLC (solvent: DCM/MeOH = 95:5, Polygram ALOX) R_{f}: 0,62

| example | structure | Name |
|---|---|---|
| 16/20 | 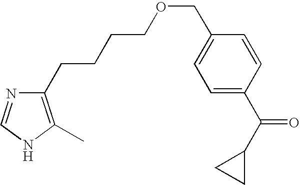 | Cyclopropyl- {4-[4-(5- methyl-1H- imidazol-4- yl)-butoxy- methyl]- phenyl}- methanone |

Example 16/20 has been prepared staffing from example 5 and p-cyclopropyl-carbonyl-benzylbromide.
Mass Spectrometry: [M + H]⁺ 313
Mass Spectrometry: [M − H]⁻ 311
TLC (solvent: DCM/MeOH = 95:5, Polygram ALOX) R_{f}: 0,62

| example | structure | Name |
|---|---|---|
| 16/21 | 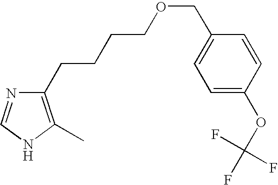 | 5-Methyl-4- [4-(4-trifluoro- methoxy- benzyloxy)- butyl]-1H- imidazole |

Example 16/21 has been prepared starting from example 5 and 4-(trifluormethoxy)-benzylbromide.
Mass Spectrometry: [M + H]⁺ 329
Mass Spectrometry: [M − H]⁻ 327
TLC (solvent: DCM/MeOH = 95:5, Polygram ALOX) R_{f}: 0,55

| example | structure | Name |
|---|---|---|
| 16/22 | 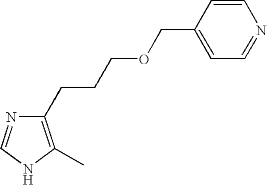 | 4-[3-(5-Methyl- 1H-imidazol-4- yl)-propoxy- methyl]-pyridine |

Example 16/22 has been prepared starting from example 1 and 4-(bromomethyl)pyridine.
Mass Spectrometry: [M + H]⁺ 232
¹H-NMR (300 MHz, CDCl₃): 1.92 (p, 2H); 2.17 (s, 3H); 2.65 (t, 2H); 3.53 (t, 2H); 4.48 (s, 2H); 7.24 (d, 2H); 7.42 (s, 1H); 8.56 (d, 2H).
TLC (solvent: DCM/MeOH = 95:5, Polygram ALOX) R_{f}: 0,44

| example | structure | Name |
|---|---|---|
| 16/23 | 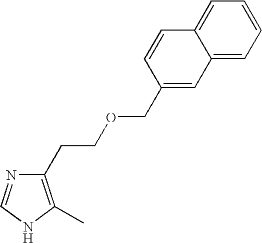 | 5-Methyl-4- [2-(naphthalen- 2-ylmethoxy)- ethyl]-1H- imidazole |

Example 16/23 has been prepared starting from example 7 and 2-(bromomethyl)-naphthalene.
Mass Spectrometry: [M + H]⁺ 267
TLC (solvent: DCM/MeOH = 95:5, Polygram ALOX) R_{f}: 0,57
MP: 88° C.

| example | structure | Name |
|---|---|---|
| 16/24 | 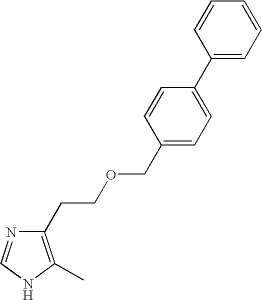 | 4-[2-(Biphenyl- 4-ylmethoxy)- ethyl]-5-methyl- 1H-imidazole |

Example 16/24 has been prepared starting from example 7 and 4-bromomethylbiphenyl.
Mass Spectrometry: [M + H]⁺ 293
Mass Spectrometry: [M − H]⁻ 291
TLC (solvent: DCM/MeOH = 95:5, Polygram ALOX) R_{f}: 0,56
MP: 109° C.

| example | structure | Name |
|---|---|---|
| 16/25 | 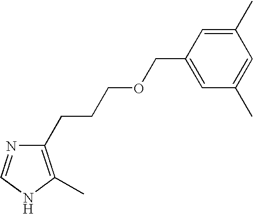 | 4-[3-(3,5-Dimethyl- benzyloxy)-propyl]- 5-methyl-1H- imidazole |

Example 16/25 has been prepared starting from example 1 and 3,5-dimethylbenzyl-bromide.
Mass Spectrometry: [M + H]⁺ 259
¹H-NMR (300 MHz, CDCl₃): 1.89 (p, 2H); 2.17 (s, 3H); 2.31 (s, 6H); 2.64 (t, 2H); 3.53 (t, 2H); 4.46 (s, 2H); 6.96 (m, 3H); 7.32 (s, 1H).
TLC (solvent: DCM/MeOH = 95:5, Polygram ALOX) R_{f}: 0,48

| example | structure | Name |
|---|---|---|
| 16/26 | 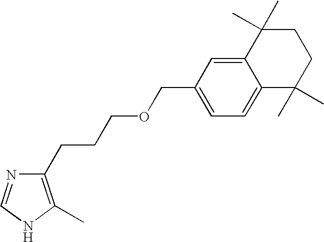 | 5-Methyl-4-[3-(5,5,8,8-tetramethyl-5,6,7,8- tetrahydro-naphthalen-2-ylmethoxy)-propyl]- 1H-imidazole |

Example 16/26 has been prepared starting from example 1 and 6-bromomethyl-1,2,3,4-tetrahydro-1,1,4,4-tetramethylnaphthalene.
Mass Spectrometry: [M + H]⁺ 341
Mass Spectrometry: [M − H]⁻ 339
TLC (solvent: DCM/MeOH = 95:5, Polygram ALOX) R_{f}: 0,56

| exam- | | |
|---|---|---|
| ple | structure | Name |
| 16/27 | 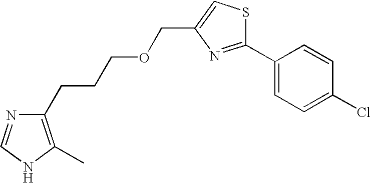 | 2-(4-Chloro-phenyl)-4-[3-(5-methyl-1H- imidazol-4-yl)-propoxymethyl]-thiazole |

Example 16/27 has been prepared starting from example 1 and 4-(chlorormethyl)-2-(4-chlororphenyl)thiazole.
Mass Spectrometry: [M + H]⁺ 384
¹H-NMR (300 MHz, CDCl₃): 1.89 (p, 2H); 2.16 (s, 3H); 2.73 (t, 2H); 3.56 (t, 2H); 4.62 (s, 2H); 7.22 (s, 1H); 7.30 (s, 1H); 7.41 (d, 2H); 7.87 (d, 2H).
TLC (solvent: DCM/MeOH = 95:5, Polygram ALOX) R_{f}: 0.56
MP: 128° C.

| exam- | | |
|---|---|---|
| ple | Structure | Name |
| 16/28 | 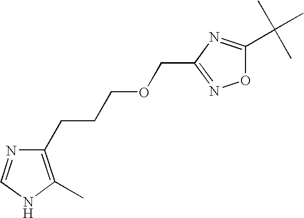 | 5-tert-Butyl-3-[3-(5-methyl-1H-imidazol-4- yl)-propoxymethyl]-[1,2,4]oxadiazole |

Example 16/28 has been prepared starting from example 1 and 5-(tert-butyl)-3-(chloromethyl)-1,2,4-oxadiazole.
Mass Spectrometry: [M + H]⁺ 280
¹H-NMR (300 MHz, CDCl₃): 1.45 (s, 9H); 1.88 (p, 2H); 2.17 (s, 3H); 2.70 (t, 2H); 3.60 (t, 2H); 4.61 (s, 2H); 7.44 (s, 1H).

| exam- | | |
|---|---|---|
| ple | Structure | Name |
| 16/29 | 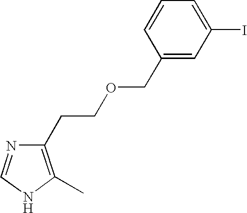 | 4-[2-(3-Iodo-benzyloxy)-ethyl]-5-methyl-1H- imidazole |

Example 16/29 has been prepared starting from example 7 and 3-iodobenzylbromide.
Mass Spectrometry: [M + H]⁺ 343
Mass Spectrometry: [M − H]⁻ 341
TLC (solvent: DCM/MeOH = 95:5, Polygram ALOX) R_{f}: 0.51

| exam- | | |
|---|---|---|
| ple | structure | Name |
| 16/30 | 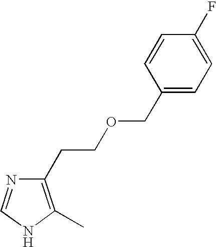 | 4-[2-(4-Fluoro-benzyloxy)-ethyl]-5-methyl- 1H-imidazole |

Example 16/30 has been prepared starting from example 7 and 4-fluorobenzylbromide.
Mass Spectrometry: [M + H]⁺ 235
Mass Spectrometry: [M − H]⁻ 233
TLC (solvent: DCM/MeOH = 95:5, Polygram ALOX) R_{f}: 0.49
MP: 55° C.

| exam- | | |
|---|---|---|
| ple | structure | Name |
| 16/31 | 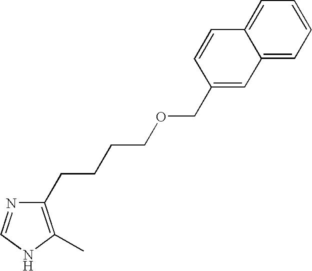 | 5-Methyl-4-[4-(naphthalen-2-ylmethoxy)- butyl]-1H-imidazole |

Example 16/31 has been prepared starting from example 5 and 2-(bromomethyl)-naphthalene.
Mass Spectrometry: [M + H]⁺ 296
Mass Spectrometry: [M − H]⁻ 294
TLC (solvent: DCM/MeOH = 95:5, Polygram ALOX) R_{f}: 0.46

| exam- | | |
|---|---|---|
| ple | structure | Name |
| 16/32 | 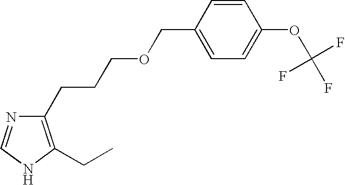 | 5-Ethyl-4-[3-(4-trifluoromethoxy-benzyloxy)- propyl]-1H-imidazole |

Example 16/32 has been prepared starting from example 9 and 4-(trifluormethoxy)-benzylbromide.
Mass Spectrometry: [M + H]⁺ 329
Mass Spectrometry: [M − H]⁻ 327
TLC (solvent: DCM/MeOH = 95:5, Polygram ALOX) R_{f}: 0.57

| exam- | | |
|---|---|---|
| ple | structure | Name |
| 16/33 | 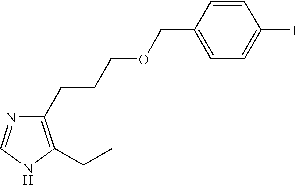 | 5-Ethyl-4-[3-(4-iodo-benzyloxy)-propyl]-1H- imidazole |

Example 16/33 has been prepared starting from example 9 and and 4-iodobenzylbromide.
Mass Spectrometry: [M + H]⁺ 371
Mass Spectrometry: [M − H]⁻ 369
TLC (solvent: DCM/MeOH = 95:5, Polygram ALOX) R_{f}: 0.54

| exam- | | |
|---|---|---|
| ple | structure | Name |
| 16/34 | 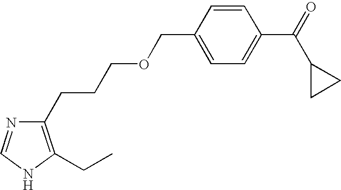 | Cyclopropyl-{4-[3-(5-ethyl-1H-imidazol-4- yl)-propoxymethyl]-phenyl}-methanone |

Example 16/34 has been prepared starting from example 9 and p-cyclopropyl-carbonyl-benzylbromide.
Mass Spectrometry: [M + H]⁺ 313
Mass Spectrometry: [M − H]⁻ 311
TLC (solvent: DCM/MeOH = 95:5, Polygram ALOX) R_{f}: 0.56
MP: 93° C.

| exam- | | |
|---|---|---|
| ple | structure | Name |
| 16/35 | 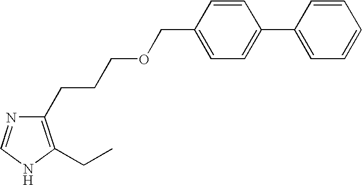 | 4-[3-(Biphenyl-4-ylmethoxy)-propyl]-5-ethyl- 1H-imidazole |

Example 16/35 has been prepared starting from example 9 and 4-bromomethylbiphenyl.
Mass Spectrometry: [M + H]⁺ 322
Mass Spectrometry: [M − H]⁻ 319
TLC (solvent: DCM/MeOH = 95:5, Polygram ALOX) R_{f}: 0.56
MP: 123° C.

| exam- | | |
|---|---|---|
| ple | structure | Name |
| 16/36 | 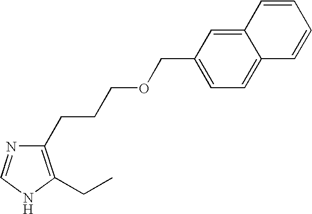 | 5-Ethyl-4-[3-(naphthalen-2-ylmethoxy)- propyl]-1H-imidazole |

Example 16/36 has been prepared starting from example 9 and 2-(bromomethyl)-naphthalene.
Mass Spectrometry: [M + H]⁺ 295
Mass Spectrometry: [M − H]⁻ 294
TLC (solvent: DCM/MeOH = 95:5, Polygram ALOX) R_{f}: 0.58
MP: 78° C.

| exam- | | |
|---|---|---|
| ple | structure | Name |
| 16/37 | 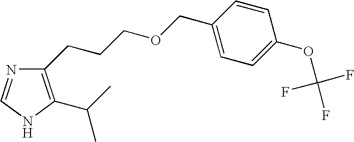 | 5-Isopropyl-4-[3-(4-trifluoromethoxy- benzyloxy)-propyl]-1H-imidazole |

Example 16/37 has been prepared starting from example 11 and 4-(trifluormethoxy)-benzylbromide.
Mass Spectrometry: [M + H]⁺ 343
Mass Spectrometry: [M − H]⁻ 341
TLC (solvent: DCM/MeOH = 95:5, Polygram ALOX) R_{f}: 0,58

| exam- | | |
|---|---|---|
| ple | structure | Name |
| 16/38 | 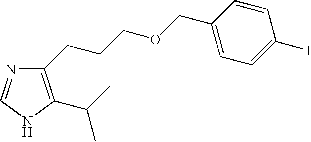 | 4-[3-(4-Iodo-benzyloxy)-propyl]-5-isopropyl-1H-imi- dazole |

Example 16/38 has been prepared starting from example 11 and 4-iodobenzyl-bromide.
Mass Spectrometry: [M + H]⁺ 385
Mass Spectrometry: [M − H]⁻ 383

| exam- | | |
|---|---|---|
| ple | structure | Name |
| 16/39 | 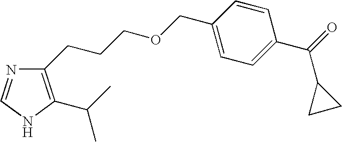 | Cyclopropyl-{4-[3-(5-isopropyl-1H-imidazol-4-yl)-propoxy- methyl]-phenyl}-methanone |

Example 16/39 has been prepared starting from example 11 and p-cyclopropyl-carbonyl-benzylbromide.
Mass Spectrometry: [M + H]⁺ 327
Mass Spectrometry: [M − H]⁻ 326
TLC (solvent: DCM/MeOH = 95:5, Polygram ALOX) R_{f}: 0,53

| exam- | | |
|---|---|---|
| ple | structure | Name |
| 16/40 | 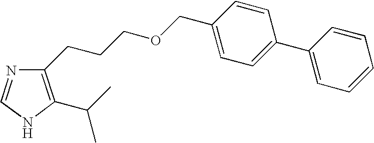 | 4-[3-(Biphenyl-4-ylmethoxy)-propyl]-5-iso- propyl-1H-imidazole |

Example 16/40 has been prepared starting from example 11 and 4-bromomethylbiphenyl.
Mass Spectrometry: [M + H]⁺ 335
Mass Spectrometry: [M − H]⁻ 333
TLC (solvent: DCM/MeOH = 95:5, Polygram ALOX) R_{f}: 0,54

| exam- | | |
|---|---|---|
| ple | structure | Name |
| 16/41 | 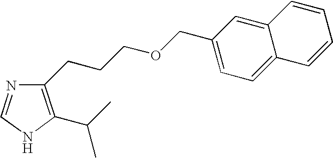 | 5-Isopropyl-4-[3-(naphthalen-2-ylmethoxy)-pro- pyl]-1H-imidazole |

Example 16/41 has been prepared starting from example 11 and 2-(bromomethyl)-naphthalene.
Mass Spectrometry: [M + H]⁺ 309
Mass Spectrometry: [M − H]⁻ 307
TLC (solvent: DCM/MeOH = 95:5, Polygram ALOX) R_{f}: 0,55
MP: 106° C.

| exam- | | |
|---|---|---|
| ple | structure | Name |
| 16/42 | 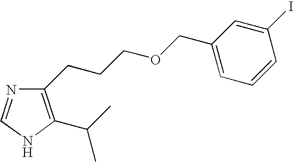 | 4-[3-(3-Iodo-benzyloxy)-propyl]-5-isopropyl-1H-imi- dazole |

Example 16/42 has been prepared starting from example 11 and 3-iodobenzylbromide.
Mass Spectrometry: [M + H]⁺ 385
Mass Spectrometry: [M − H]⁻ 383
TLC (solvent: DCM/MeOH = 95:5, Polygram ALOX) R_{f}: 0,48

| exam- | | |
|---|---|---|
| ple | structure | Name |
| 16/43 | 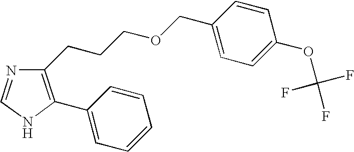 | 5-Phenyl-4-[3-(4-trifluormethoxy-benzyl- oxy)-propyl]-1H-imidazole |

Exmaple 16/43 has been prepared starting from example 13 and 4-(trifluoromethoxy)-benzylbromide.
Mass Spectrometry: [M + H]⁺ 377
Mass Spectrometry: [M − H]⁻ 375
TLC (solvent: DCM/MeOH = 95:5, Polygram ALOX) R_{f}: 0,61
MP: 105° C.

| exam- | | |
|---|---|---|
| ple | structure | Name |
| 16/44 | 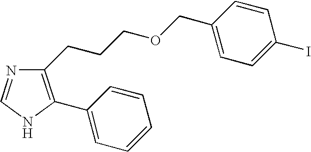 | 4-[3-(4-Iodo-benzyloxy)-propyl]-5-phenyl-1H-imi- dazole |

Example 16/44 has been prepared starting from example 13 and 4-iodobenzylbromide.
Mass Spectrometry: [M + H]⁺ 419
Mass Spectrometry: [M − H]⁻ 417
TLC (solvent: DCM/MeOH = 95:5, Polygram ALOX) R_{f}: 0,70
MP: 64° C.

| exam- | | |
|---|---|---|
| ple | Structure | Name |
| 16/45 | 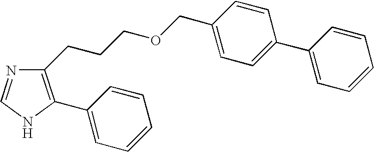 | 4-[3-(Biphenyl-4-ylmethoxy)-propyl]-5-phen- yl-1H-imidazole |

Example 16/45 has been prepared starting from example 13 and 4-bromomethylbiphenyl.
Mass Spectrometry: [M + H]⁺ 369
Mass Spectrometry: [M − H]⁻ 367
TLC (solvent: DCM/MeOH = 95:5, Polygram ALOX) R_{f}: 0,59
MP: 133° C.

| exam- | | |
|---|---|---|
| ple | Structure | Name |
| 16/46 | 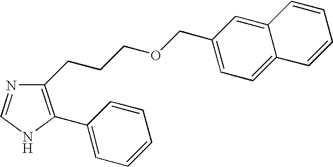 | 4-[3-(Naphthalen-2-ylmethoxy)-propyl]-5-phen- yl-1H-imidazole |

Example 16/46 has been prepared starting from example 13 and 2-(bromomethyl)-naphthalene.
Mass Spectrometry: [M + H]⁺ 343
Mass Spectrometry: [M − H]⁻ 341
TLC (solvent: DCM/MeOH = 95:5, Polygram ALOX) R_{f}: 0,59
MP: 112° C.

| example | structure | Name |
|---|---|---|
| 16/47 | 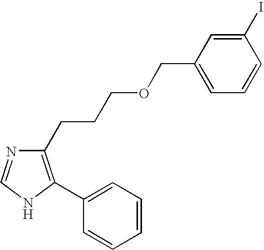 | 4-[3-(3-Iodo- benzyloxy)- propyl]-5-phenyl- 1H-imidazole |

Example 16/47 has been prepared starting from example 13 and 3-iodobenzylbromide.
Mass Spectrometry: [M + H]⁺ 419
Mass Spectrometry: [M − H]⁻ 417
TLC (solvent: DCM/MeOH = 95:5, Polygram ALOX) R_{f}: 0,58
MP: 67° C.

### Example 17

### 1-{4-[3-(5-Methyl-1H-imidazol-4-yl)-propoxy]-phenyl}-ethanone oxime

To a mixtures of 1 mmol of example 3/3, 750 mg pyridine and 25 ml ethanol 650 mg hydroxylamine hydrochlorid were added. The mixture was stirred and refluxed for three hours. After removal of the solvents under reduced pressure, H₂O and EtOAc were added. The crude product was extracted and the organic layer was dried (MgSO₄) before evaporation of the solvent. The residue was chromatographed on SiO₂ using a gradient of CHCl₃ to CHCl₃/MeOH (5:1) yielding the product as solids.

| example | structure | Name |
|---|---|---|
| 17 | 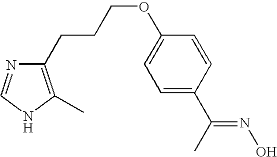 | 1-{4-[3- (5-Methyl-1H- imidazol-4-yl)- propoxy]- phenyl}- ethanone oxime |

Mass Spectrometry: [M + H]⁺ 274
Mass Spectrometry: [M − H]⁺ 272
¹H-NMR (300 MHz, CDCl₃): 1.86-2.1 (m, 8H); 2.5-2.65 (m, 2H); 3.85-3.94 (m, 2H); 6.87-6.91 (d, 2H); 7.51-7.53 (d, 2H); 7.32 (s, 1H); 10.95 (s, 1H); 11.5-11.57 (d, 1H).
TLC (solvent: DCM/MeOH = 95:5, Polygram ALOX) R_{f}: 0,29
MP: 154° C.

### Example 18

### Tablet Containing 50 mg of Active Substance

A typical tablet, which may be prepared by conventional tabletting techniques, may contain:

Tablet containing 50 mg of active substance
Composition:

| | |
|---|---|
| (1) Active substance | 50.0 mg |
| (2) Lactose | 98.0 mg |
| (3) Maize starch | 50.0 mg |
| (4) Polyvinylpyrrolidone | 15.0 mg |
| (5) Magnesium stearate | 2.0 mg |
| | 215.0 mg |

Preparation:
(1), (2) and (3) are mixed together and granulated with an aqueous
solution of (4). (5) is added to the dried granulated material.
From this mixture tablets are pressed, biplanar, faceted on both
sides and with a dividing notch on one side.
Diameter of the tablets: 9 mm.

### Example 19

### Capsules Containing 50 mg of Active Substance

A typical hard gelatine capsule, which may be prepared by conventional techniques, may contain:

Capsules containing 50 mg of active substance
Composition:

| | |
|---|---|
| (1) Active substance | 50.0 mg |
| (2) Dried maize starch | 58.0 mg |
| (3) Powdered lactose | 50.0 mg |
| (4) Magnesium stearate | 2.0 mg |
| | 160.0 mg |

Preparation:
(1) is triturated with (3). This trituration is added to
the mixture of (2) and (4) with vigorous mixing.

This powder mixture is packed into size 3 hard gelatine capsules in a capsule filling machine.

### Example 20

### Suppositories Containing 150 mg of Active Substance

Typical suppositories may contain:

Suppositories containing 150 mg of active substance:
1 suppository contains:

| | |
|---|---|
| Active substance | 150.0 mg |
| Polyethylenglycol 1500 | 550.0 mg |
| Polyethylenglycol 6000 | 460.0 mg |
| Polyethylenesorbitan monostearate | 840.0 mg |
| | 2000.0 mg |

Preparation:
The polyethyleneglycol is melted together with
polyethylenesorbitan monostearate. The ground active
substance is homogeneously dispersed in the melt.
This is then poured into slightly chilled suppository moulds.

### Example 21

### Dry Ampoule Containing 35 mg of Active Substance per 2 ml Composition

Typical dry ampoules may contain:

Dry ampoule containing 35 mg of active substance per 2 ml
Composition:

| | | |
|---|---|---|
| Active substance | 35.0 | mg |
| Mannitol | 100.0 | mg |
| water for injections | ad 2.0 | ml |

Preparation:
Active substance and mannitol are dissolved in water. After packaging, the
solution is freeze-dried.

To produce the solution ready for use, the product is dissolved in water for injections.

1. A compound of formula (I): 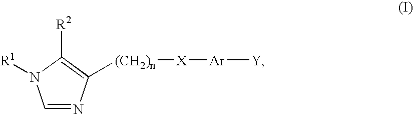
wherein
R¹ is a hydrogen atom or a functional group which can convert into a hydrogen atom in vivo,
R² is a C₁₋₆-alkyl, C₃₋₇-cycloalkyl, aryl or aryl-C₁₋₂-alkyl group,
n is 2, 3, 4 or 5,
X is an oxygen or sulfur atom or a —CO—, —O—CH₂— or —SO—CH₂— group,
Ar is a phenylene or naphthylene group,
a 5-membered heteroarylene group linked via a carbon or nitrogen atom and containing
an imino group optionally substituted by a C₁₋₄-alkyl or C₁₋₄-alkyl-carbonyl group, or an oxygen or sulfur atom,
an imino group optionally substituted by a C₁₋₄-alkyl group, or an oxygen or sulfur atom, and additionally a nitrogen atom,
an imino group optionally substituted by a C₁₋₄-alkyl group, and two nitrogen atoms or
an oxygen or sulfur atom, and two nitrogen atoms,
or a 6-membered heteroarylene group containing one or two nitrogen atoms,
wherein the above-mentioned phenylene or 5- or 6-membered hetero-arylene groups are optionally condensed via pairs of two adjacent carbon atoms with one or two saturated, unsaturated or aromatic carbocyclic or heterocyclic groups, each of which are optionally substituted by one or two carbonyl or C₁₋₃-alkyl groups,
and the resulting condensed bi- or tricycles may be linked to X via the carbocyclic or heterocyclic moiety, and
Y is a hydrogen, fluorine, chlorine, bromine or iodine atom, a hydroxy, cyano, C₁₋₆-alkyl, C₃₋₇-cycloalkyl, acetylene, C₁₋₄-alkyl-acetylene, C₁₋₄-alkyl-carbonyl, C₃₋₇-cycloalkyl-carbonyl, —C(═N—OH)—CH₃, phenyl, 5- or 6-membered heteroaryl, C₁₋₆-alkyloxy or phenyloxy group,
wherein the phenyl rings contained in all the above definitions may additionally be substituted by one or two fluorine, chlorine, bromine or iodine atoms, or by one or two C₁₋₆-alkyl or C₁₋₆-alkoxy groups, wherein the substituents may be the same or different, and
the hydrogen atoms of alkyl groups contained in all the above definitions may be partly or fully replaced by fluorine atoms,
or a diastereomer, enantiomer, mixture or salt thereof.

2. A compound of formula I according to claim 1, wherein
R¹ is a hydrogen atom or a trityl group,
R² is a C₁₋₄-alkyl, C₃₋₅-cycloalkyl or aryl group,
n is 2, 3 or 4,
X is an oxygen or sulfur atom or a —O—CH₂— or —SO—CH₂— group,
Ar is a 1,2-phenylene, 1,3-phenylene, 1,4-phenylene, 2,5-naphthylene or 2,6-naphthylene group,
a 5-membered heteroarylene group linked via a carbon or nitrogen atom and a carbon atom containing
an imino group optionally substituted by a C₁₋₄-alkyl or C₁₋₄-alkyl-carbonyl group, or an oxygen or sulfur atom,
an imino group optionally substituted by a C₁₋₄-alkyl group, or an oxygen or sulfur atom, and additionally a nitrogen atom,
an imino group optionally substituted by a C₁₋₄-alkyl group, and two nitrogen atoms or
an oxygen or sulfur atom, and two nitrogen atoms,
or a 6-membered heteroarylene group containing one or two nitrogen atoms,
wherein two adjacent carbon atoms of the above-mentioned phenylene or 5- or 6-membered heteroarylene groups are optionally bridged by a —CH₂—CH₂—CH₂—CH₂—, —(C═O)—CH₂—CH₂—CH₂—, —C(CH₃)₂—CH₂—CH₂—C(CH₃)₂—, —CH═CH—CH═N—, —O—CH₂—O— or —N═CH—S— group;
and the resulting bicycles are linked to X via the carbocyclic moiety, and
Y is a hydrogen, fluorine, chlorine, bromine or iodine atom, a hydroxy, cyano, C₁₋₄-alkyl, C₃₋₅-cycloalkyl, acetylene, C₁₋₄-alkyl-acetylene, C₁₋₄-alkyl-carbonyl, C₃₋₅-cycloalkyl-carbonyl, phenyl, —C(═N—OH)—CH₃, C₁₋₆-alkyloxy, phenyloxy group or a 5- or 6-membered heteroaryl group as defined above,
wherein the phenyl rings contained in all the above definitions may additionally be substituted by one or two halogen atoms, C₁₋₆-alkyl or C₁₋₆-alkoxy groups while the substituents may be the same or different and
the hydrogen atoms of alkyl groups contained in all the above definitions may be partly or fully replaced by fluorine atoms,
or a diastereomer, enantiomer, mixture or salt thereof.

3. A compound according to claim 2, wherein
R¹, R², n, Ar and Y are as defined in claim 2 and
X is an oxygen or sulfur atom or a —O—CH₂— group
or a diastereomer, enantiomer, mixture, or salt thereof.

4. A compound of formula I according to claim 1, wherein
R¹ is a hydrogen atom,
R² is a C₁₋₄-alkyl, C₃₋₅-cycloalkyl or phenyl group,
n is 2, 3 or 4,
X is an oxygen atom or a —O—CH₂— group,
Ar is a group selected from the formulae 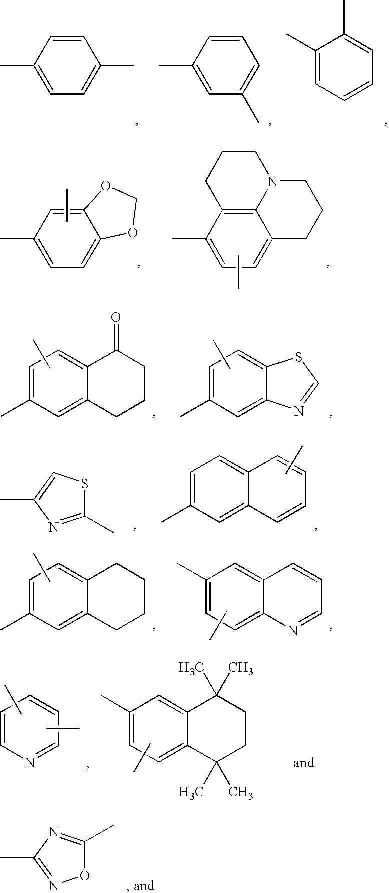
Y is a hydrogen, fluorine, chlorine, bromine or iodine atom, a hydroxy, cyano, C₁₋₄-alkyl, C₃₋₅-cycloalkyl, acetylene, C₁₋₄-alkyl-carbonyl, C₃₋₅-cycloalkyl-carbonyl, phenyl, —C(═N—OH)—CH₃, C₁₋₃-alkoxy, phenyloxy or imidazolyl group,
wherein the phenyl rings contained in all the above definitions may additionally be substituted by a halogen atom, a C₁₋₃-alkyl or C₁₋₃-alkoxy group and
the hydrogen atoms of alkyl groups contained in all the above definitions may be partly or fully replaced by fluorine atoms,
or a diastereomer, enantiomer, mixture or salt thereof.

5. A compound of formula I according to claim 4, wherein
R¹ is a hydrogen atom,
R² is a methyl, ethyl or isopropyl group,
n is 2, 3 or 4,
X is an oxygen atom or a —O—CH₂— group,
Ar is a 1,3- or 1,4-phenylene or 2,5-naphthylene group and
Y is a hydrogen, fluorine, chlorine, bromine or iodine atom, a hydroxy, cyano, C₁₋₄-alkyl, acetylene, C₁₋₄-alkyl-carbonyl, C₃₋₅-cycloalkyl-carbonyl, phenyl, C₁₋₃-alkoxy, phenoxy or imidazolyl group,
wherein the phenyl rings contained in all the above definitions may additionally be substituted by a fluorine, chlorine, bromine or iodine atom atom, a C₁₋₃-alkyl or C₁₋₃-alkoxy group and
the hydrogen atoms of alkyl groups contained in all the above definitions may be partly or fully replaced by fluorine atoms,
or a diastereomer, enantiomer, mixture or salt thereof.

6. A compound of formula I according to claim 5 wherein
R² is a methyl group
or a diastereomer, enantiomer, mixture or salt thereof.

7. A compound of formula I according to claim 1 selected from the following compounds:
(a) 5-Methyl-4-[4-(naphthalen-2-yloxy)-butyl]-1H-imidazole,
(b) 4-[3-(4-Iodo-benzyloxy)-propyl]-5-methyl-1H-imidazole,
(c) 5-Methyl-4-[3-(4-trifluoromethoxy-benzyloxy)-propyl]-1H-imidazole,
(d) 5-Methyl-4-[3-(naphthalen-2-ylmethoxy)-propyl]-1H-imidazole,
(e) 5-Methyl-4-[3-(4-trifluoromethyl-benzyloxy)-propyl]-1H-imidazole,
(f) 4-[3-(3,5-Dichloro-benzyloxy)-propyl]-5-methyl-1H-imidazole,
(g) 4-[3-(3,5-Bis-trifluoromethyl-benzyloxy)-propyl]-5-methyl-1H-imidazole,
(h) 4-[3-(3-Iodo-benzyloxy)-propyl]-5-methyl-1H-imidazole,
(i) 5-Methyl-4-[3-(3-trifluoromethyl-benzyloxy)-propyl]-1H-imidazole,
(j) 4-[2-(4-Iodo-benzyloxy)-ethyl]-5-methyl-1H-imidazole,
(k) 5-Methyl-4-[4-(4-trifluoromethoxy-benzyloxy)-butyl]-1H-imidazole and
(l) 4-[3-(3,5-Dimethyl-benzyloxy)-propyl]-5-methyl-1H-imidazole,
or a diastereomer, enantiomer, mixture or salt thereof.

8. A pharmaceutically acceptable salt of a compound according to claim 1.

9. A pharmaceutical composition comprising a compound according to claim 1.

10. A pharmaceutical composition comprising a pharmaceutically acceptable salt according to claim 8.

11. A method of treating ischemic arrhythmia, myocardial ischemia and infarction, asthma, chronic vasomotor rhinitis or pain, or a method for gastroprotective therapy, which comprises administering to a patient in need thereof a therapeutically effective amount of a compound according to claim 1.
